(19) United States
(12) Patent Application Publication
(10) Pub. No.: US 2019/0364241 A1
(43) Pub. Date: Nov. 28, 2019

(54) INFORMATION RECORDING SYSTEM, INFORMATION RECORDING DEVICE, AND INFORMATION RECORDING METHOD

(71) Applicant: OLYMPUS CORPORATION, Tokyo (JP)

(72) Inventors: Yoshihiro KAWANO, Tokyo (JP); Seiji TATSUTA, Tokyo (JP)

(73) Assignee: OLYMPUS CORPORATION, Tokyo (JP)

(21) Appl. No.: 16/535,362

(22) Filed: Aug. 8, 2019

Related U.S. Application Data
(63) Continuation of application No. PCT/JP2018/003139, filed on Jan. 31, 2018.

(30) Foreign Application Priority Data
Feb. 14, 2017 (JP) 2017-025068

(51) Int. Cl. H04N 5/77; H04N 7/18; G06K 7/14; G06K 19/06

(52) U.S. Cl. CPC H04N 5/77; H04N 7/18; G06K 19/06037; G06K 7/1417; G06K 19/06028; G06K 7/1413

(57) ABSTRACT

In an information recording system, an information generation unit is configured to generate processing information for identifying processing to be performed on an object for inspection. An ID acquisition unit is configured to acquire ID information attached to at least one of the object and a physical object for storing or supporting the object. A recording unit is configured to associate first ID information and second ID information with the processing information and record the processing information, the first ID information, and the second ID information on a recording medium. The first ID information is the ID information acquired by the ID acquisition unit before the processing is performed. The second ID information is the ID information acquired by the ID acquisition unit after the processing is performed.

The present application claims priority to Japanese Patent Application No. 2017-025068, filed on Feb. 14, 2017 and is a continuation application based on PCT Patent Application No. PCT/JP2018/003139, filed on Jan. 31, 2018, and the content of both the Japanese patent application and the PCT patent application is incorporated herein by reference.

### BACKGROUND OF THE INVENTION

### Field of the Invention

The present invention relates to an information recording system, an information recording device, and an information recording method.

### Description of Related Art

At an inspection site, a process of division, processing, and the like is performed on a sample which is an inspection object. The inspection is performed on the processed sample. For example, in pathological diagnosis, diagnosis is performed on the basis of a pathological specimen made from tissue such as a patient's organ. In situations in which a large number of samples are required to be processed, there is a possibility that the samples will be mixed up.

Technology for reducing the number of times samples are mixed up is disclosed in Japanese Unexamined Patent Application, First Publication No. 2013-072693. In this technology, a first feature quantity generated from an image of a sample is recorded on an IC tag. The IC tag on which the first feature quantity is recorded is added to a first container for storing the sample. When the sample is moved from the first container to a second container, the sample is photographed and a second feature quantity is generated from an image of the sample. It is determined whether or not the first feature quantity recorded on the IC tag matches the second feature quantity. Thereby, sample matching is performed.

### SUMMARY OF THE INVENTION

According to a first aspect of the present invention, an information recording system includes an information generation unit, an ID acquisition unit, and a recording unit. The information generation unit includes at least one of a sensor and a camera. The information generation unit generates processing information for identifying processing to be performed on an object for inspection. The ID acquisition unit includes at least one of a communicator and a scanner. The ID acquisition unit acquires ID information attached to at least one of the object and a physical object for storing or supporting the object. The recording unit includes a processor. The recording unit associates first ID information and second ID information with the processing information and records the processing information, the first ID information, and the second ID information on a recording medium. The first ID information is the ID information acquired by the ID acquisition unit before the processing is performed. The second ID information is the ID information acquired by the ID acquisition unit after the processing is performed.

According to a second aspect of the present invention, in the first aspect, the information generation unit may be mounted on an instrument for use in the processing and output the processing information when the use of the instrument has been detected.

According to a third aspect of the present invention, in the second aspect, the instrument may be used to make pathological specimens.

According to a fourth aspect of the present invention, in the first aspect, the information generation unit may include a camera configured to generate image information by photographing a state of the processing. The recording unit may record the image information generated by the camera as the processing information on the recording medium.

According to a fifth aspect of the present invention, in the first aspect, the information recording system may further include a first instruction acceptance unit including at least one of an operation device and a communicator and configured to accept a start instruction related to a start of the processing and an end instruction related to an end of the processing. The first ID information may be the ID information acquired by the ID acquisition unit at a timing associated with a timing at which the start instruction is accepted. The second ID information may be the ID information acquired by the ID acquisition unit at a timing associated with a timing at which the end instruction is accepted.

According to a sixth aspect of the present invention, in the first aspect, the ID acquisition unit may include a first ID acquisition unit and a second ID acquisition unit including at least one of a communicator and a scanner. The first ID acquisition unit may acquire the first ID information. The second ID acquisition unit may be different from the first ID acquisition unit and acquire the second ID information.

According to a seventh aspect of the present invention, in the first aspect, the ID acquisition unit may include a camera configured to generate image information by photographing at least one of the object and the physical object. The recording unit may record the image information generated by the camera as the ID information on the recording medium.

According to an eighth aspect of the present invention, in the first aspect, the ID acquisition unit may include a camera and an image processing unit including a processor. The camera generates image information by photographing at least one of the object and the physical object. The image processing unit acquires the ID information by performing image processing on the image information generated by the camera.

According to a ninth aspect of the present invention, in the eighth aspect, the image processing unit may acquire the first ID information from a first region in the image information. The image processing unit may acquire the second ID information from a second region different from the first region in the image information.

According to a tenth aspect of the present invention, in the eighth aspect, when a first motion of the ID information in the image information has been recognized, the image processing unit may acquire the ID information as the first ID information. When a second motion of the ID information in the image information has been recognized, the image processing unit may acquire the ID information as the second ID information. The second motion may be different from the first motion.

According to an eleventh aspect of the present invention, in the first aspect, a radio tag on which the ID information is recorded may be attached to at least one of the object and the physical object. The ID acquisition unit may be a wireless communicator configured to receive the ID information from the radio tag.

According to a twelfth aspect of the present invention, in the first aspect, the ID acquisition unit may include a sound acquisition unit including a microphone and a sound processing unit including a processor. The sound acquisition unit acquires sound information based on sound uttered by a user who performs the processing. The sound processing unit recognizes the ID information on the basis of the sound information acquired by the sound acquisition unit.

According to a thirteenth aspect of the present invention, in the first aspect, the information generation unit may generate the processing information corresponding to each of a plurality of types of the processing. The ID acquisition unit may acquire the first ID information and the second ID information corresponding to each of the plurality of types of the processing. The recording unit may associate the first ID information and the second ID information corresponding to each of the plurality of types of the processing with the processing information corresponding to each of the plurality of types of the processing. The recording unit may record the processing information, the first ID information, and the second ID information corresponding to each of the plurality of types of the processing on the recording medium.

According to a fourteenth aspect of the present invention, in the first aspect, the recording unit may further associate time point information indicating a time point at which the processing was performed with the processing information and record the time point information on the recording medium.

According to a fifteenth aspect of the present invention, in the first aspect, the information recording system may further include a second instruction acceptance unit including at least one of an operation device and a communicator and a reading unit including a processor. The second instruction acceptance unit accepts an instruction for specifying at least one piece of the first ID information and the second ID information. The reading unit reads the processing information associated with at least one piece of the first ID information and the second ID information corresponding to the instruction from the recording medium and reads the first ID information and the second ID information associated with the processing information from the recording medium.

According to a sixteenth aspect of the present invention, in the fifteenth aspect, when the second instruction acceptance unit has accepted an instruction for specifying the second ID information, the reading unit may read first processing information associated with the second ID information corresponding to the instruction from the recording medium and read the first ID information and the second ID information associated with the first processing information from the recording medium. The reading unit may further read second processing information associated with the second ID information identical to the first ID information associated with the first processing information from the recording medium and read the first ID information and the second ID information associated with the second processing information from the recording medium.

According to a seventeenth aspect of the present invention, in the fifteenth aspect, when the second instruction acceptance unit has accepted an instruction for specifying the first ID information, the reading unit may read first processing information associated with the first ID information corresponding to the instruction from the recording medium and read the first ID information and the second ID information associated with the first processing information from the recording medium. The reading unit may further read second processing information associated with the first ID information identical to the second ID information associated with the first processing information from the recording medium and read the first ID information and the second ID information associated with the second processing information from the recording medium.

According to an eighteenth aspect of the present invention, in the first aspect, the information recording system may further include a third instruction acceptance unit including at least one of an operation device and a communicator and a reading unit including a processor. The third instruction acceptance unit accepts an instruction for specifying the processing information. The reading unit reads the processing information corresponding to the instruction from the recording medium and read the first ID information and the second ID information associated with the processing information from the recording medium.

According to a nineteenth aspect of the present invention, in the eighteenth aspect, when the third instruction acceptance unit has accepted an instruction for specifying first processing information, the reading unit may read the first processing information corresponding to the instruction from the recording medium and read the first ID information and the second ID information associated with the first processing information from the recording medium. The reading unit may further read second processing information associated with the second ID information identical to the first ID information associated with the first processing information from the recording medium and read the first ID information and the second ID information associated with the second processing information from the recording medium.

According to a twentieth aspect of the present invention, in the eighteenth aspect, when the third instruction acceptance unit has accepted an instruction for specifying first processing information, the reading unit may read the first processing information corresponding to the instruction from the recording medium and read the first ID information and the second ID information associated with the first processing information from the recording medium. The reading unit may further read second processing information associated with the first ID information identical to the second ID information associated with the first processing information from the recording medium and read the first ID information and the second ID information associated with the second processing information from the recording medium.

According to a twenty-first aspect of the present invention, in the fifteenth or eighteenth aspect, the information recording system may further include a display configured to display the processing information, the first ID information, and the second ID information read by the reading unit.

According to a twenty-second aspect of the present invention, in the first aspect, the information recording system may further include a determination unit including a processor and a warning unit including at least one of a display and a speaker. The determination unit determines whether or not at least one piece of the processing information, the first ID information, and the second ID information recorded on the recording medium matches a condition predefined as a condition to be satisfied by the processing information, the first ID information, and the second ID information. The warning unit issues a warning when at least one piece of the processing information, the first ID information, and the second ID information recorded on the recording medium does not match the condition.

According to a twenty-third aspect of the present invention, an information recording device includes an input unit including at least one of an input terminal and a communicator and a recording unit including a processor. Processing information generated by an information generation unit including at least one of a sensor and a camera and ID information acquired by an ID acquisition unit including at least one of a communicator and a scanner are input to the input unit. The information generation unit generates the processing information for identifying processing to be performed on an object for inspection. The ID acquisition unit acquires the ID information attached to at least one of the object and a physical object for storing or supporting the object. The recording unit associates first ID information and second ID information with the processing information and records the processing information, the first ID information, and the second ID information on a recording medium. The first ID information is the ID information acquired by the ID acquisition unit before the processing is performed. The second ID information is the ID information acquired by the ID acquisition unit after the processing is performed.

According to a twenty-fourth aspect of the present invention, an information recording method includes an information generation step, an ID acquisition step, and a recording step. In the information generation step, an information generation unit including at least one of a sensor and a camera generates processing information for identifying processing to be performed on an object for inspection. In the ID acquisition step, an ID acquisition unit including at least one of a communicator and a scanner acquires ID information attached to at least one of the object and a physical object for storing or supporting the object. In the recording step, a recording unit including a processor associates first ID information and second ID information with the processing information and records the processing information, the first ID information, and the second ID information on a recording medium. The first ID information is the ID information acquired by the ID acquisition unit before the processing is performed. The second ID information is the ID information acquired by the ID acquisition unit after the processing is performed.

According to a twenty-fifth aspect of the present invention, an information recording method includes an input step and a recording step. In the input step, processing information generated by an information generation unit including at least one of a sensor and a camera and ID information acquired by an ID acquisition unit including at least one of a communicator and a scanner are input to an input unit including at least one of an input terminal and a communicator. The information generation unit generates the processing information for identifying processing to be performed on an object for inspection. The ID acquisition unit acquires the ID information attached to at least one of the object and a physical object for storing or supporting the object. In the recording step, a recording unit including a processor associates first ID information and second ID information with the processing information and records the processing information, the first ID information, and the second ID information on a recording medium. The first ID information is the ID information acquired by the ID acquisition unit before the processing is performed. The second ID information is the ID information acquired by the ID acquisition unit after the processing is performed.

### BRIEF DESCRIPTION OF THE DRAWINGS

FIG. 1 is a block diagram showing a configuration of an information recording system according to a first embodiment of the present invention.

FIG. 2 is a flowchart showing order of a process to be performed by the information recording system according to the first embodiment of the present invention.

FIG. 3 is a conceptual diagram showing an example of information to be recorded by the information recording system according to the first embodiment of the present invention.

FIG. 4 is a conceptual diagram showing an example of information to be recorded by the information recording system according to the first embodiment of the present invention.

FIG. 5 is a reference diagram showing an example of a time relationship between a plurality of types of processing in the first embodiment of the present invention.

FIG. 6 is a block diagram showing a configuration of an information recording system according to a first modified example of the first embodiment of the present invention.

FIG. 7 is a flowchart showing order of a process to be performed by the information recording system according to the first modified example of the first embodiment of the present invention.

FIG. 8 is a block diagram showing a configuration of an information recording system according to a second modified example of the first embodiment of the present invention.

FIG. 9 is a block diagram showing a configuration of an information recording system according to a third modified example of the first embodiment of the present invention.

FIG. 10 is a block diagram showing a configuration of an information recording system according to a fourth modified example of the first embodiment of the present invention.

FIG. 11 is a flowchart showing order of a process to be performed by the information recording system according to the fourth modified example of the first embodiment of the present invention.

FIG. 12 is a reference diagram showing an example of image information generated in the fourth modified example of the first embodiment of the present invention.

FIG. 13 is a schematic diagram of a site where processing is performed on an object in the fourth modified example of the first embodiment of the present invention.

FIG. 14 is a reference diagram showing an example of image information generated in the fourth modified example of the first embodiment of the present invention.

FIG. 15 is a block diagram showing a configuration of an information recording system according to a fifth modified example of the first embodiment of the present invention.

FIG. 16 is a block diagram showing a configuration of an information recording system according to a second embodiment of the present invention.

FIG. 17 is a flowchart showing order of a process to be performed by the information recording system according to the second embodiment of the present invention.

FIG. 18 is a reference diagram showing an example of information to be read by the information recording system according to the second embodiment of the present invention.

FIG. 19 is a reference diagram showing an example of information to be read by the information recording system according to the second embodiment of the present invention.

FIG. 20 is a reference diagram showing an example of information to be read by the information recording system according to the second embodiment of the present invention.

FIG. 21 is a flowchart showing order of a process to be performed by an information recording system according to a third embodiment of the present invention.

FIG. 22 is a reference diagram showing an example of information to be read by the information recording system according to the third embodiment of the present invention.

FIG. 23 is a reference diagram showing an example of information to be read by the information recording system according to the third embodiment of the present invention.

FIG. 24 is a reference diagram showing an example of information to be read by the information recording system according to the third embodiment of the present invention.

FIG. 25 is a block diagram showing a configuration of an information recording system according to a fourth embodiment of the present invention.

FIG. 26 is a flowchart showing order of a process to be performed by the information recording system according to the fourth embodiment of the present invention.

FIG. 27 is a reference diagram showing an example of pathological diagnosis in a fifth embodiment of the present invention.

FIG. 28 is a reference diagram showing an example of pathological diagnosis in the fifth embodiment of the present invention.

FIG. 29 is a reference diagram showing an example of pathological diagnosis in the fifth embodiment of the present invention.

FIG. 30 is a reference diagram showing an example of pathological diagnosis in the fifth embodiment of the present invention.

FIG. 31 is a block diagram showing a configuration of an information recording system according to a sixth embodiment of the present invention.

FIG. 32 is a flowchart showing order of a process to be performed by the information recording device according to the sixth embodiment of the present invention.

FIG. 33 is a block diagram showing a configuration of an information recording system according to a seventh embodiment of the present invention.

FIG. 34 is a flowchart showing order of a process to be performed by the information recording device according to the seventh embodiment of the present invention.

FIG. 35 is a block diagram showing a configuration of an information recording system according to an eighth embodiment of the present invention.

### DETAILED DESCRIPTION OF THE INVENTION

Hereinafter, embodiments of the present invention will be described with reference to the drawings.

### First Embodiment

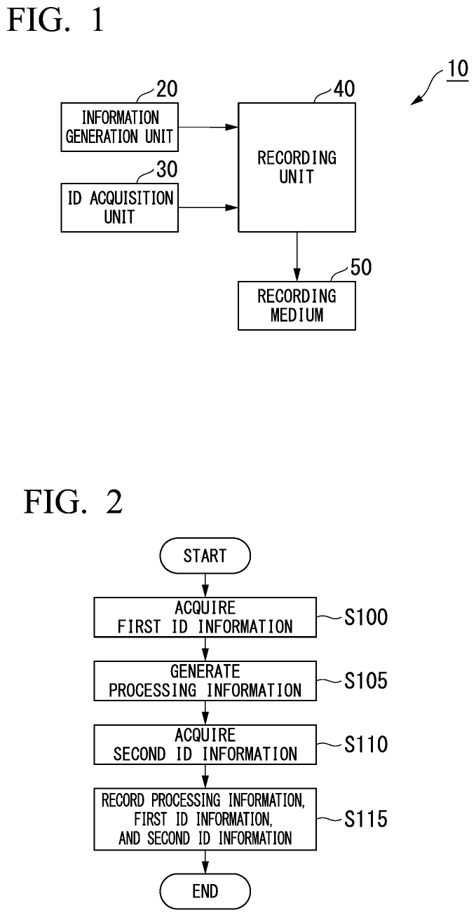

FIG. 1 shows a configuration of an information recording system 10 according to a first embodiment of the present invention. As shown in FIG. 1, the information recording system 10 includes an information generation unit 20, an ID acquisition unit 30, a recording unit 40, and a recording medium 50.

A schematic configuration of the information recording system 10 will be described. The information generation unit 20 generates processing information for identifying processing to be performed on an object. The ID acquisition unit 30 acquires ID information attached to at least one of an object and a physical object for storing or supporting the object. The recording unit 40 associates first ID information and second ID information with the processing information and records the processing information, the first ID information, and the second ID information on the recording medium 50. The first ID information is the ID information acquired by the ID acquisition unit 30 before the processing is performed. The second ID information is the ID information acquired by the ID acquisition unit 30 after the processing is performed.

A detailed configuration of the information recording system 10 will be described. An object is a physical object that is a target of processing constituting each step in an inspection. For example, a physical object for storing or supporting the object is a container for use in various types of processing. The physical object for storing or supporting the object may be a cartridge or a glass slide to be used to make a pathological specimen. In the following description, the container is mainly used as the physical object for storing or supporting the object. The container used in the following description can be replaced with a physical object such as a cartridge or a glass slide according to processing details.

For example, the information generation unit 20 is mounted on an instrument used for the processing and outputs processing information when the use of the instrument has been detected. This processing information can be used to identify a type of processing. For example, the information generation unit 20 includes a sensor. For example, the sensor is installed in an instrument for applying a reagent to the object.

For example, the sensor is a pressure sensor or an acceleration sensor. When the pressure generated by holding the instrument is detected by the pressure sensor, the information generation unit 20 outputs the processing information. Alternatively, when the acceleration sensor detects acceleration generated by the movement of the instrument, the information generation unit 20 outputs the processing information. The processing information according to a type of instrument, i.e., processing details, is output from the information generation unit 20. The instrument may be a device configured to automatically perform the processing on the object. The information generation unit 20 may be a sensor configured to detect the execution of the processing by the device. For example, the information generation unit 20 outputs the processing information when a start button of the device configured to perform the processing on the object is pushed.

The instrument on which the information generation unit 20 is mounted may be used to make a pathological specimen. For example, the instrument may be an embedding device, a slicing device (a microtome), a staining device, or a coverslipper. The coverslipper is an instrument for attaching a cover glass to a glass slide.

The information generation unit 20 may include a camera configured to generate image information by photographing a state of processing. The recording unit 40 may record the image information generated by the camera as the processing information on the recording medium 50. The camera includes an imaging element (an image sensor). The camera photographs at least the object. The camera is disposed to include such that at least the object is included in a photographing range. The image information indicates a state of the object corresponding to processing details. The camera may photograph the object and the surroundings of the object. In this case, the camera is disposed such that the object and the surroundings of the object are included in the photographing range. For example, the surroundings of the object include at least a portion of a user who performs the processing. The surroundings of the object may include at least a portion of a device used for the processing. The image information may be either moving-image information or still-image information. ID information acquired by the ID acquisition unit 30 may be added as tag information to the image information.

The camera may be a wearable camera worn by the user. The wearable camera is a head-mounted camera mounted near the eyes of an observer such that image information according to a viewpoint of the observer can be acquired. Therefore, the camera may be disposed at a position of the user's viewpoint or a position near the user's viewpoint.

When the information generation unit 20 is a camera, the information recording system 10 can easily generate the processing information including processing details. When the information generation unit 20 is a wearable camera, the information recording system 10 can easily generate the processing information including the processing details acquired from the user's viewpoint.

For example, a radio tag on which the ID information is recorded is attached to at least one of the object and the container. The ID acquisition unit 30 is a wireless communication unit (wireless communicator) configured to receive the ID information from the radio tag. The radio tag is an IC chip including a memory and a wireless communication unit. The ID acquisition unit 30 is a reader in a radio frequency identifier (RFID). The ID information is recorded in the memory. The wireless communication unit included in the radio tag and the ID acquisition unit 30 includes a radio circuit such as an oscillator and an antenna. The ID information is information for identifying at least one of the object and the container.

A code such as a barcode or a two-dimensional code may be attached as the ID information to at least one of the object and the container. The ID acquisition unit 30c may be a code scanner (a code reader) configured to read a code.

When the ID information is attached only to the object, the ID information is acquired from the object. When the ID information is attached only to the object, first ID information and second ID information may be identical. When the ID information is attached only to the container, the ID information is acquired from the container. When the ID information is attached only to the container and the same container is used before and after the processing, the first ID information and the second ID information may be identical. When different ID information is attached to each of the object and the container, the ID information is a combination of ID information acquired from the object and ID information acquired from the container.

For example, the recording unit 40 includes a recording processing circuit configured to perform an information recording process. At least one piece of the processing information and the ID information may be compressed. Therefore, the recording unit 40 may include a compression processing circuit for compressing information. The recording unit 40 may include a buffer for a recording process and a compression process. The processing information generated by the information generation unit 20 and the ID information acquired by the ID acquisition unit 30 are input to the recording unit 40. The ID information acquired by the ID acquisition unit 30 before the processing is performed is input as the first ID information to the recording unit 40. After the processing is performed, the ID information acquired by the ID acquisition unit 30 is input as the second ID information to the recording unit 40. The recording unit 40 records the processing information, the first ID information, and the second ID information associated with each other on the recording medium 50.

The recording unit 40 may include one or more processors. For example, the processor is at least one of a central processing unit (CPU), a digital signal processor (DSP), and a graphics processing unit (GPU). The recording unit 40 may include an application specific integrated circuit (ASIC) or a field-programmable gate array (FPGA).

For example, one file including the processing information, the first ID information, and the second ID information is generated and therefore the respective information is associated. A name of the file including data of the processing information may include information such as character strings for identifying the first ID information and the second ID information. When a plurality of pieces of information recorded on the recording medium 50 are associated with each other, other pieces of information associated with one piece of information can be identified on the basis of the one piece of information.

The recording medium 50 is a non-volatile storage device. For example, the recording medium 50 is at least one of an erasable programmable read only memory (EPROM), an electrically erasable programmable read-only memory (EEPROM), a flash memory, and a hard disk drive. The recording medium 50 may not be installed at an inspection site. For example, the information recording system 10 may include a network interface and the information recording system 10 may be connected to the recording medium 50 via networks such as the Internet and a local area network (LAN). The information recording system 10 may include a wireless communication interface and the information recording system 10 may be connected to the recording medium 50 by wireless communication according to standards such as Wi-Fi (registered trademark) and Bluetooth (registered trademark). Therefore, the information recording system 10 may not directly include the recording medium 50.

The information recording system 10 may include a notification unit. For example, the notification unit is a display unit such as a liquid crystal display. For example, the display unit is a monitor of a personal computer (PC). The display unit may be a wearable display such as smart glasses worn by the user. The display unit may be a light emitting device such as a light emitting diode (LED). The notification unit may be a speaker.

For example, the notification unit instructs the user to read the first ID information before the processing on the object is started. The user causes the ID acquisition unit 30 to read the first ID information. The ID acquisition unit 30 acquires the first ID information. After the first ID information is acquired, the notification unit instructs the user to process the object. When a predetermined time has elapsed or after the end of the processing is detected, the notification unit instructs the user to read the second ID information. The user causes the ID acquisition unit 30 to read the second ID information. The ID acquisition unit 30 acquires the second ID information. In this case, the recording unit 40 records the ID information acquired by the ID acquisition unit 30 during a first period as the first ID information on the recording medium 50. Also, the recording unit 40 records the ID information acquired by the ID acquisition unit 30 during a second period after the first period as the second ID information on the recording medium 50.

FIG. 2 shows order of a process to be performed by the information recording system 10. The order of the process to be performed by the information recording system 10 will be described with reference to FIG. 2.

The ID acquisition unit 30 acquires first ID information (step S100 (ID acquisition step)). After step S100, the information generation unit 20 generates processing information (step S105 (information generation step)). After step S105, the ID acquisition unit 30 acquires second ID information (step S110 (ID acquisition step)). After step S110, the recording unit 40 associates the first ID information and the second ID information with the processing information and records the processing information, the first ID information, and the second ID information on the recording medium 50 (step S115 (recording step)).

As described above, the processing information, the first ID information, and the second ID information are associated with each other and recorded on the recording medium 50. The processing details can be identified on the basis of the processing information. The first ID information corresponds to the object before the processing is performed. The second ID information corresponds to the object after the processing is performed. Therefore, other information associated with any one of the object and the processing details can be identified from any one of the object and the processing details. Thereby, the information recording system 10 can support verification of the validity of a processing situation.

At an inspection site where a large number of objects are handled, the objects can be mixed up. For example, the mix-up of the objects occurs when the user stores the object in a wrong container. The user can check whether or not the mix-up of the objects has occurred by checking the processing information associated with the ID information corresponding to the object. For example, when the processing information is image information, the user checks a situation of an operation performed by the user using an image. Thereby, the user can check a cause of the mix-up of the objects. When a plurality of types of processing are performed on the object, the user can ascertain a relationship between the plurality of types of processing on the basis of the ID information. Therefore, the user can identify a plurality of types of processing performed on a specific object. The user can check whether or not the mix-up of the objects has occurred on the basis of the processing information corresponding to each type of processing.

When the processing information is image information, the user can check whether or not the processing details are correct by checking the image. When the processing information is information for identifying a type of processing, the user can check whether or not the type of processing performed on the object is correct. Therefore, the user can verify the validity of the processing details on the basis of the processing information.

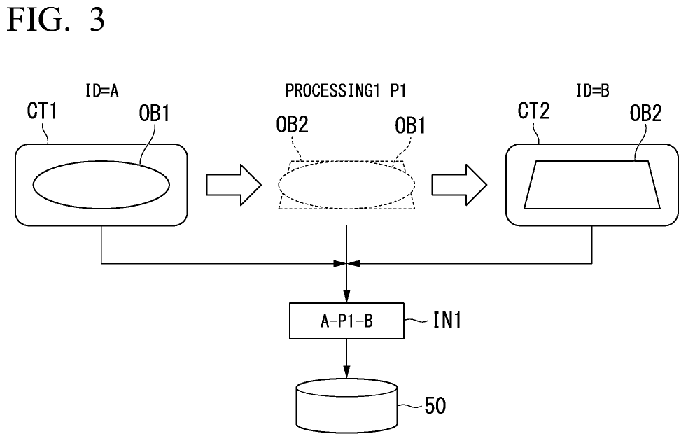

FIG. 3 shows an example of information to be recorded by the information recording system 10. An example of a process to be performed by the information recording system 10 will be described with reference to FIG. 3.

Before processing 1 is performed, an object OB1 is stored in a container CT1. First ID information A is attached to the container CT1. The ID acquisition unit 30 acquires the first ID information A attached to the container CT1. The information generation unit 20 generates processing information P1 corresponding to the processing 1. An object OB2 is generated by performing the processing 1 on the object OB1. After the processing 1 is performed, the object OB2 is stored in a container CT2. Second ID information B is attached to the container CT2. The ID acquisition unit 30 acquires the second ID information B attached to the container CT2. The recording unit 40 associates the first ID information A and the second ID information B with the processing information P1. The recording unit 40 records information IN1 in which the processing information P1, the first ID information A, and the second ID information B are associated with each other on the recording medium 50.

The information IN1 includes the processing information and the ID information associated with each other. For example, the first ID information A is shown on the left side of the processing information P1 and the second ID information B is shown on the right side of the processing information P1. A straight line between the processing information P1 and the first ID information A and a straight line between the processing information P1 and the second ID information B indicate that the respective information is associated. Also in the drawings other than FIG. 3, the association of information is shown in a format similar to that of the information IN1. The recording format is not limited thereto. Any recording format can be used as long as the association is known. As in a normal database, these pieces of information may be recorded as one record in accordance with a predetermined format.

A plurality of types of processing may be sequentially performed on the object. The information generation unit 20 generates processing information corresponding to each of the plurality of types of processing. The ID acquisition unit 30 acquires the first ID information and the second ID information corresponding to each of the plurality of types of processing. The recording unit 40 associates the first ID information and the second ID information corresponding to each of the plurality of types of processing with the processing information of each of the plurality of types of processing. The recording unit 40 records the processing information, the first ID information, and the second ID information corresponding to each of the plurality of types of processing on the recording medium 50.

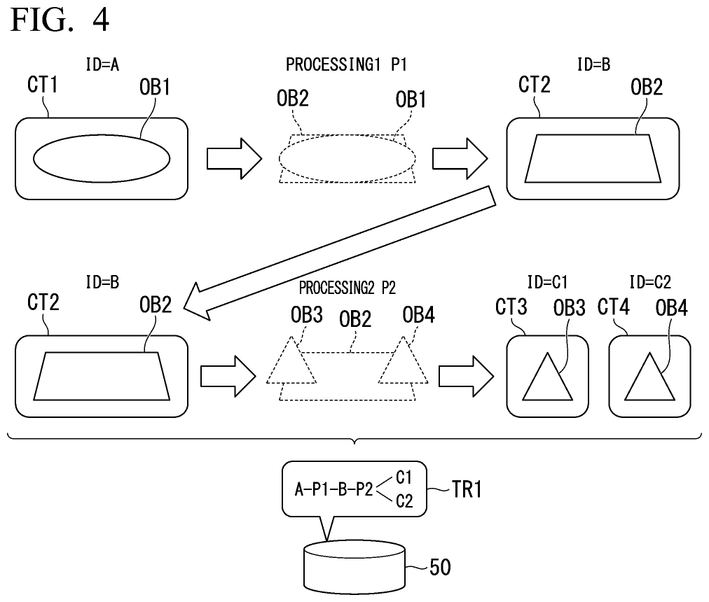

FIG. 4 shows an example of information to be recorded by the information recording system 10. In FIG. 4, an example in which a plurality of types of processing are sequentially performed on an object is shown. An example of a process to be performed by the information recording system 10 will be described with reference to FIG. 4.

Processing 1 and processing 2 are performed on the object. Processing performed by the information recording system 10 in relation to the processing 1 is similar to the processing shown in FIG. 3. Before the processing 2 is performed, an object OB2 is stored in a container CT2. First ID information B is attached to the container CT2. The ID acquisition unit 30 acquires the first ID information B attached to the container CT2. The information generation unit 20 generates processing information P2 corresponding to the processing 2. An object OB3 and an object OB4 are generated by performing the processing 2 on the object OB2.

After the processing 2 is performed, the object OB3 is stored in a container CT3. Second ID information C1 is attached to the container CT3. The ID acquisition unit 30 acquires the second ID information C1 attached to the container CT3. The recording unit 40 associates the first ID information B and the second ID information C1 with processing information P2. The recording unit 40 records the processing information P2, the first ID information B, and the second ID information C1 in association on the recording medium 50. After the processing 2 is performed, the object OB4 is stored in a container CT4. Second ID information C2 is attached to the container CT4. The ID acquisition unit 30 acquires the second ID information C2 attached to the container CT4. The recording unit 40 associates the first ID information B and the second ID information C2 with the processing information P2. The recording unit 40 records the processing information P2, the first ID information B, and the second ID information C2 in association on the recording medium 50.

A tree TR1 indicates a relationship of the information recorded on the recording medium 50. Information related to the processing 1 includes the processing information P1, the first ID information A, and the second ID information B. Information related to the processing 2 includes the processing information P2, the first ID information B, the second ID information C1, and the second ID information C2. When the object is divided into a plurality of objects by performing the processing, the recording unit 40 commonly associates the second ID information corresponding to each of the plurality of objects with the processing information. For example, in the above-described example, the recording unit 40 commonly associates the second ID information C1 and the second ID information C2 with the processing information P2.

As shown in the tree TR1, a relationship between the plurality of types of processing performed on the object is recorded. The user can ascertain the relationship between the plurality of types of processing on the basis of the information recorded on the recording medium 50. Therefore, the user can identify a plurality of types of processing performed on a specific object. The user can verify the validity of a processing process on the basis of the processing information corresponding to each type of processing.

The recording unit 40 may associate time point information indicating a time point at which the processing was performed with the processing information and record the time point information on the recording medium 50.

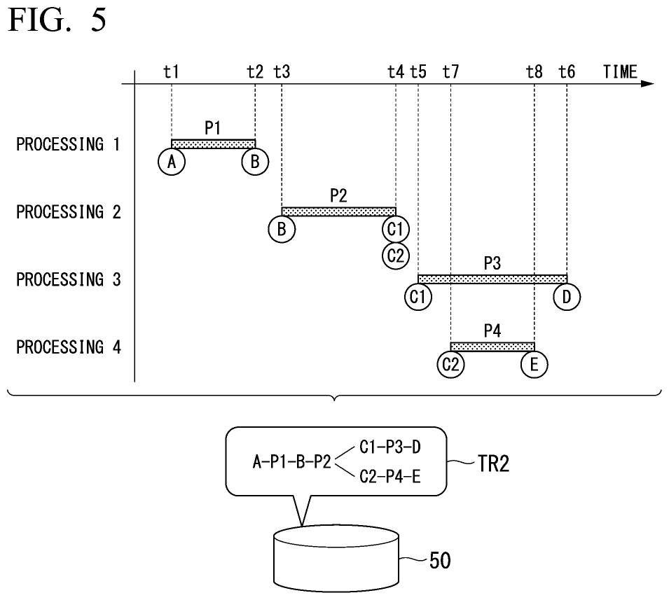

FIG. 5 shows an example of a time relationship between a plurality of types of processing. A third example of information to be recorded by the information recording system 10 will be described with reference to FIG. 5.

In FIG. 5, a period in which processing was performed on an object is shown for each type of processing. The horizontal direction in FIG. 5 represents time. A start time point of processing 1 is t1 and an end time point of the processing 1 is t2. First ID information A and second ID information B are associated with processing information P1 of the processing 1. A start time point of processing 2 is t3 and an end time point of the processing 2 is t4. The time point t3 is later than the time point t2. Two objects are generated by the processing 2. First ID information B, second ID information C1, and second ID information C2 are associated with processing information P2 of the processing 2. A start time point of processing 3 is t5 and an end time point of the processing 3 is t6. The time point t5 is later than the time point t4. First ID information C1 and second ID information D are associated with processing information P3 of the processing 3. A start time point of processing 4 is t7, and an end time point of the processing 4 is t8. The time point t7 is later than the time points t4 and t5 and is earlier than the time point t6. The time point t8 is earlier than the time point t6. That is, a period in which the processing 4 is performed overlaps a period in which the processing 3 is performed. First ID information C2 and second ID information E are associated with processing information P4 of the processing 4.

The recording unit 40 records the processing information P1, the first ID information A, the second ID information B, the time point information t1, and the time point information t2 related to the processing 1 on the recording medium 50. The recording unit 40 records the processing information P2, the first ID information B, the second ID information C1, the second ID information C2, the time point information t3, and the time point information t4 related to the processing 2 on the recording medium 50. The recording unit 40 records the processing information P3, the first ID information C1, the second ID information D, the time point information t5, and the time point information t6 related to the processing 3 on the recording medium 50. The recording unit 40 records the processing information P4, the first ID information C2, the second ID information E, the time point information t7, and the time point information t8 related to the processing 4 on the recording medium 50. For example, when the processing information is image information, the time point information is added as tag information to the processing information. A tree TR2 indicates a relationship between the processing information and the ID information recorded on the recording medium 50.

For example, the container for storing the object before the processing is performed may be the same as the container for storing the object after the processing is performed. Even when the same container is used before and after each of a plurality of successive processes, the information recording system 10 facilitates identification of the order of processing on the object by recording time point information.

In the first embodiment, the recording unit 40 associates the first ID information and the second ID information with the processing information and records the processing information, the first ID information, and the second ID information on the recording medium 50. Thus, the information recording system 10 can support verification of the validity of the processing. When the processing information is image information, the user can verify the validity of the processing details.

When a plurality of types of processing are sequentially performed on the object, the information generation unit 20 generates processing information corresponding to each of the plurality of types of processing. Thus, the user can verify the validity of the processing process on the basis of the processing information corresponding to each type of processing.

When the plurality of types of processing are sequentially performed on the object, the ID acquisition unit 30 acquires first ID information and second ID information corresponding to each of the plurality of types of processing. Therefore, the user can ascertain a relationship between the plurality of types of processing on the basis of the ID information corresponding to each type of processing.

In one example of the first embodiment, the recording unit 40 associates time point information indicating a time point at which the processing is performed with processing information and records the time point information on the recording medium 50. Thereby, the information recording system 10 facilitates identification of the order of processing on the object.

### First Modified Example of First Embodiment

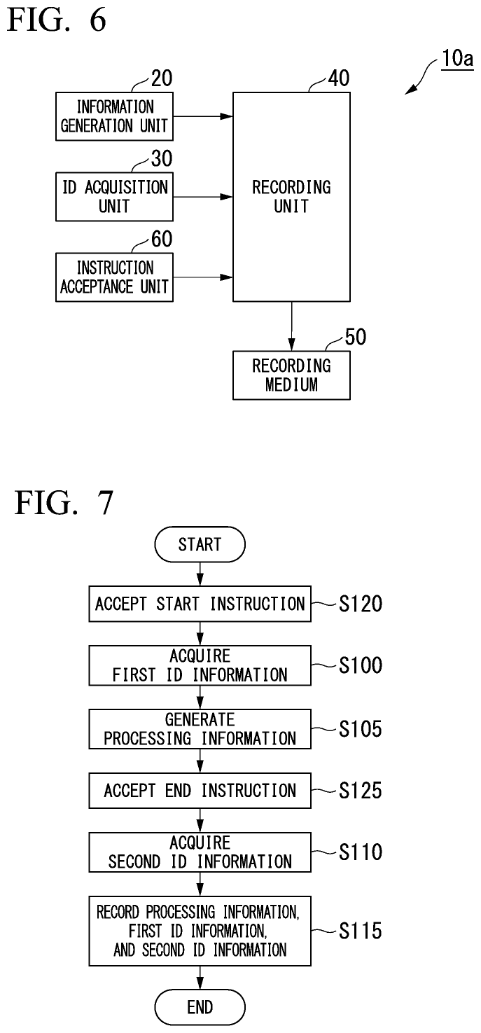

FIG. 6 shows a configuration of an information recording system 10a according to a first modified example of the first embodiment of the present invention. Differences from the configuration shown in FIG. 1 will be described in terms of the configuration shown in FIG. 6.

The information recording system 10a has an instruction acceptance unit 60 (a first instruction acceptance unit) in addition to the configuration of the information recording system 10 shown in FIG. 1. The instruction acceptance unit 60 accepts a start instruction related to a start of processing and an end instruction related to an end of the processing. First ID information is ID information acquired by an ID acquisition unit 30 at a timing associated with a timing at which the start instruction is accepted. Second ID information is ID information acquired by the ID acquisition unit 30 at a timing associated with a timing at which the end instruction is accepted.

For example, the instruction acceptance unit 60 is configured as an operation unit (operation device). For example, the operation unit includes at least one of a button, a switch, a key, a mouse, a joystick, a touch pad, a trackball, and a touch panel. The operation unit accepts an instruction when a predetermined operation has been performed by the user. The instruction acceptance unit 60 may be configured as a wireless communication unit configured to perform wireless communication with the operation unit. The wireless communication unit includes a radio circuit such as an oscillator and an antenna. The wireless communication unit accepts the instruction when information indicating the instruction has been received. The instruction acceptance unit 60 may include a first instruction acceptance unit configured to accept a start instruction and a second instruction acceptance unit configured to accept an end instruction.

For example, a recording unit 40 recognizes the ID information acquired by the ID acquisition unit 30 as the first ID information from a timing at which the instruction acceptance unit 60 accepts the start instruction to a timing at which the instruction acceptance unit 60 accepts the end instruction. The recording unit 40 may recognize the ID information acquired by the ID acquisition unit 30 as the first ID information within a predetermined time from the timing at which the instruction acceptance unit 60 accepts the start instruction. The ID acquisition unit 30 may acquire a plurality of pieces of ID information and the recording unit 40 may recognize the plurality of pieces of ID information as a plurality of pieces of first ID information.

For example, the recording unit 40 recognizes the ID information acquired by the ID acquisition unit 30 as the second ID information after the instruction acceptance unit 60 accepts the end instruction. The recording unit 40 may recognize the ID information acquired by the ID acquisition unit 30 as the second ID information within a predetermined time from the timing at which the instruction acceptance unit 60 accepts the end instruction. The ID acquisition unit 30 may acquire a plurality of pieces of ID information and the recording unit 40 may recognize the plurality of pieces of ID information as a plurality of pieces of second ID information.

The instruction acceptance unit 60 may be integrated with the ID acquisition unit 30. The ID acquisition unit 30 outputs ID information acquired from a timing at which the instruction acceptance unit 60 accepts the start instruction to a timing at which the instruction acceptance unit 60 accepts the end instruction as the first ID information. The ID acquisition unit 30 may acquire a plurality of pieces of ID information and output the plurality of pieces of ID information as a plurality of pieces of first ID information. The ID acquisition unit 30 outputs ID information acquired after the instruction acceptance unit 60 accepts the end instruction as the second ID information. The ID acquisition unit 30 may acquire a plurality of pieces of ID information and output the plurality of pieces of ID information as a plurality of pieces of second ID information.

The recording unit 40 may associate the above-described first ID information and second ID information with processing information generated by the information generation unit 20 from the timing at which the instruction acceptance unit 60 accepts the start instruction to the timing at which the instruction acceptance unit 60 accepts the end instruction. When a plurality of types of processing are continuously performed and the information generation unit 20 continuously generates the processing information, the information recording system 10a can record the processing information based on the start instruction and the end instruction of each type of processing.

For example, the instruction acceptance unit 60 and the ID acquisition unit 30 are configured as a code scanner configured to operate in cooperation with a processing device. Information read by the code scanner before the processing device starts processing is acquired as first ID information. Also, information read by the code scanner after the processing device completes the processing is acquired as second ID information. The first ID information and the second ID information are recorded in association with the processing information.

Except for the points described above, the configuration shown in FIG. 6 is similar to the configuration shown in FIG. 1.

FIG. 7 shows order of a process to be performed by the information recording system 10a. Differences from the process shown in FIG. 2 will be described in terms of a process shown in FIG. 7.

The instruction acceptance unit 60 accepts a start instruction (step S120 (start instruction acceptance step)). After step S120, the processing in step S100 is performed. After step S105, the instruction acceptance unit 60 accepts an end instruction (step S125 (end instruction acceptance step)). After step S125, the processing in step S110 is performed.

Except for the points described above, the process shown in FIG. 7 is similar to the process shown in FIG. 2.

As described above, the instruction acceptance unit 60 accepts the start instruction and the end instruction. Thus, the ID acquisition unit 30 can reliably acquire the first ID information and the second ID information in line with the user's intention.

### Second Modified Example of First Embodiment

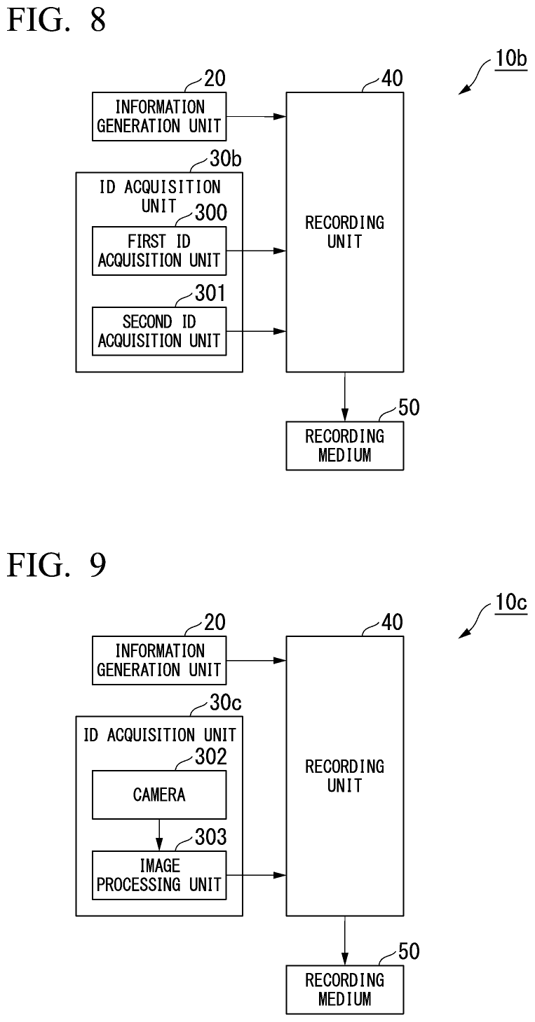

FIG. 8 shows a configuration of an information recording system 10b according to a second modified example of the first embodiment of the present invention. Differences from the configuration shown in FIG. 1 will be described in terms of the configuration shown in FIG. 8.

In the information recording system 10b, the ID acquisition unit 30 shown in FIG. 1 is changed to an ID acquisition unit 30b. The ID acquisition unit 30b includes a first ID acquisition unit 300 and a second ID acquisition unit 301. The first ID acquisition unit 300 acquires first ID information. The second ID acquisition unit 301 is different from the first ID acquisition unit 300 and acquires second ID information.

For example, the first ID acquisition unit 300 is a camera configured to read a number label attached to a container. For example, the second ID acquisition unit 301 is an RFID reader configured to read an ID from a radio tag attached to a cartridge.

Except for the points described above, the configuration shown in FIG. 8 is similar to the configuration shown in FIG. 1.

As described above, the ID acquisition unit 30b includes the first ID acquisition unit 300 and the second ID acquisition unit 301. Thereby, the ID acquisition unit 30b can independently acquire the first ID information and the second ID information.

### Third Modified Example of First Embodiment

FIG. 9 shows a configuration of an information recording system 10c according to a third modified example of the first embodiment of the present invention. Differences from the configuration shown in FIG. 1 will be described in terms of the configuration shown in FIG. 9.

In the information recording system 10c, the ID acquisition unit 30 shown in FIG. 1 is changed to an ID acquisition unit 30c. The ID acquisition unit 30c includes a camera 302 and an image processing unit 303. The camera 302 generates image information by photographing at least one of an object and a container. The image processing unit 303 acquires ID information by performing image processing on the image information generated by the camera 302. The image processing unit 303 may include one or more processors. The image processing unit 303 may include an ASIC or an FPGA.

The ID information can be visually recognized on the surface of at least one of the object and the container. For example, printed matter on which the ID information is shown is attached to the surface of at least one of the object and the container. The camera 302 generates image information in which the ID information can be visually recognized. For example, the image processing unit 303 acquires ID information by collating the image information generated by the camera 302 with the image information corresponding to predetermined ID information. For example, the image information corresponding to the predetermined ID information is pre-recorded on a recording medium 50.

A recording unit 40 may record the image information generated by the camera 302 as the ID information on the recording medium 50. In this case, the ID acquisition unit 30c may not include the image processing unit 303. For example, the recording unit 40 records image information of one frame in which the ID information is photographed as the ID information on the recording medium 50. The image information recorded on the recording medium 50 may be a thumbnail obtained by reducing the image information of the one frame. The information generation unit 20 may include a camera different from the camera 302.

Except for the points described above, the configuration shown in FIG. 9 is similar to the configuration shown in FIG. 1.

As described above, the ID acquisition unit 30c includes the camera 302. Thereby, the information recording system 10c can be easily configured.

### Fourth Modified Example of First Embodiment

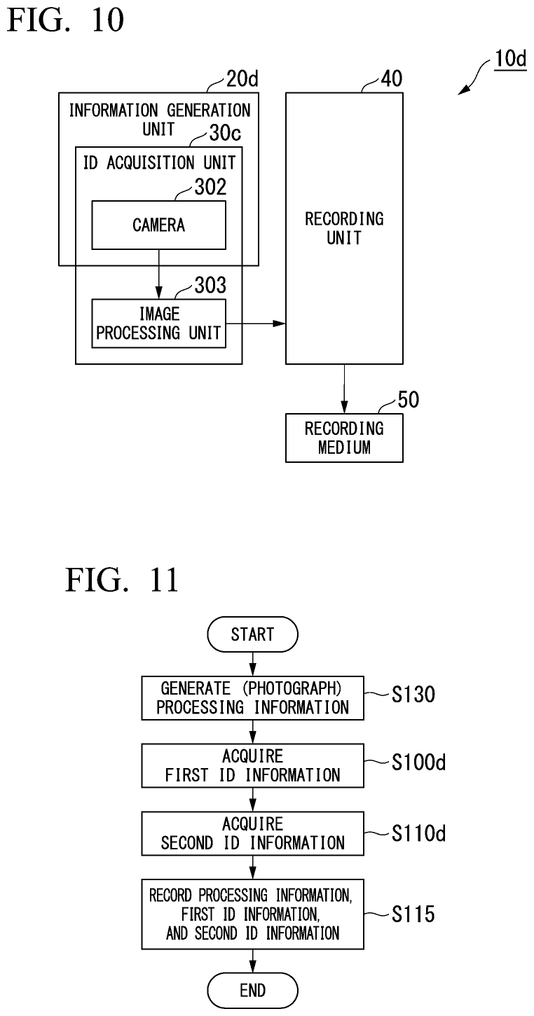

FIG. 10 shows a configuration of an information recording system 10d according to a fourth modified example of the first embodiment of the present invention. Differences from the configuration shown in FIG. 1 will be described in terms of the configuration shown in FIG. 10.

In the information recording system 10d, the information generation unit 20 shown in FIG. 1 is changed to an information generation unit 20d, and the ID acquisition unit 30 shown in FIG. 1 is changed to an ID acquisition unit 30c. The information generation unit 20d includes a camera 302. The ID acquisition unit 30c is configured like the ID acquisition unit 30c shown in FIG. 9. The camera 302 generates image information by photographing at least one of the object and the container and a state of processing. The camera 302 is shared between the information generation unit 20d and the ID acquisition unit 30c.

Except for the points described above, the configuration shown in FIG. 10 is similar to the configuration shown in FIG. 1.

FIG. 11 shows order of a process to be performed by the information recording system 10d. Differences from the process shown in FIG. 2 will be described in terms of a process shown in FIG. 11.

The camera 302 generates image information by photographing at least one of an object and a container and a state of processing (step S130 (information generation step)). For example, the camera 302 photographs the object and its surroundings and generates moving-image information. The moving-image information includes image information of a plurality of frames generated at different time points. The moving-image information generated by the camera 302 includes first to third image information. The first image information is image information of a frame in which first ID information is recorded. The second image information is image information of a frame in which a state of the processing on the object is recorded. The second image information constitutes processing information. The third image information is image information of a frame in which second ID information is recorded. The image processing unit 303 outputs the second image information as the processing information. Alternatively, the image processing unit 303 outputs the entire moving-image information including the first to third image information as the processing information.

After step S130, the image processing unit 303 acquires the first ID information by performing image processing on the image information generated by the camera 302, i.e., the first image information (step S100d (ID acquisition step)). After step S100d, the image processing unit 303 acquires the second ID information by performing image processing on the image information generated by the camera 302, i.e., the third image information (step S110d (ID acquisition step)). After step S110d, the processing in step S115 is performed.

Except for the points described above, the process shown in FIG. 11 is similar to the process shown in FIG. 2.

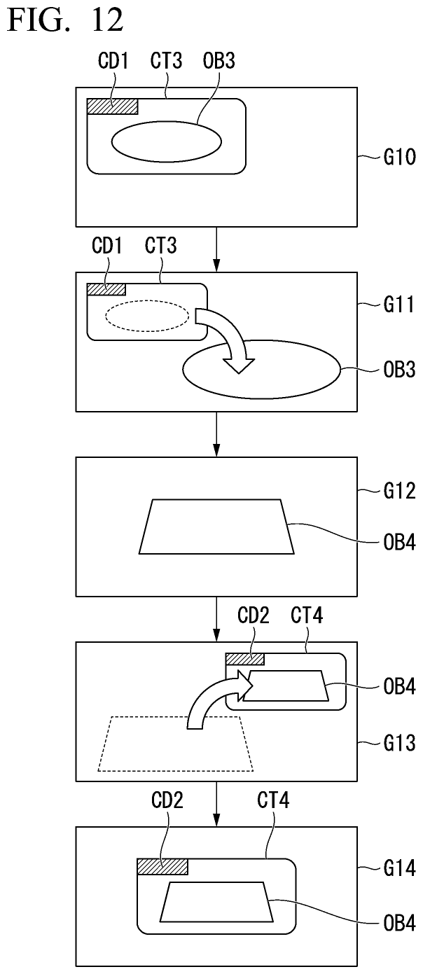

A first example in which first ID information and second ID information are generated will be described. FIG. 12 shows an example of image information generated by the camera 302. The camera 302 generates moving-image information. Image information of representative frames within image information of a plurality of frames constituting the moving-image information is shown in FIG. 12.

Image information G10 is generated before processing on an object OB3 is performed. The image information G10 shows an image of the object OB3 and a container CT3. The object OB3 is stored in the container CT3. A code CD1 corresponding to first ID information is attached to the container CT3. The image processing unit 303 acquires the first ID information by performing image processing on the image information G10. The recording unit 40 may record the image information G10 as the first ID information on the recording medium 50.

After the image information G10 is generated, image information G11 is generated. The image information G11 is image information indicating a state until the object OB3 is removed from the container CT3. In FIG. 12, an image of a site after the object OB3 is removed from the container CT3 is shown.

After the processing on the object OB3 is started, image information G12 is generated. By performing the processing on the object OB3, an object OB4 is generated. The image information G12 is image information indicating a state until the object OB4 is generated by performing the processing on the object OB3. In FIG. 12, an image after the object OB4 is generated is shown.

After the object OB4 is generated, image information G13 is generated. The image information G13 is image information indicating a state until the object OB4 is stored in a container CT4. In FIG. 12, an image of a site after the object OB4 is stored in the container CT4 is shown. A code CD2 corresponding to the second ID information is attached to the container CT4.

After the image information G13 is generated, image information G14 is generated. The image information G14 shows an image of the object OB4 and the container CT4. The image processing unit 303 acquires the second ID information by performing image processing on the image information G14. The recording unit 40 may record the image information G14 as the second ID information on the recording medium 50.

Moving-image information including the image information G10 to G14 is recorded as processing information on the recording medium 50. The moving-image information may be recorded as the processing information on the recording medium 50 only during a processing period from a timing at which the processing on the object OB3 is started to a timing at which the processing is completed and the object OB4 is generated.

A second example in which first ID information and second ID information are generated will be described. The image processing unit 303 acquires the first ID information from a first region in image information. The image processing unit 303 acquires the second ID information from a second region different from the first region in the image information.

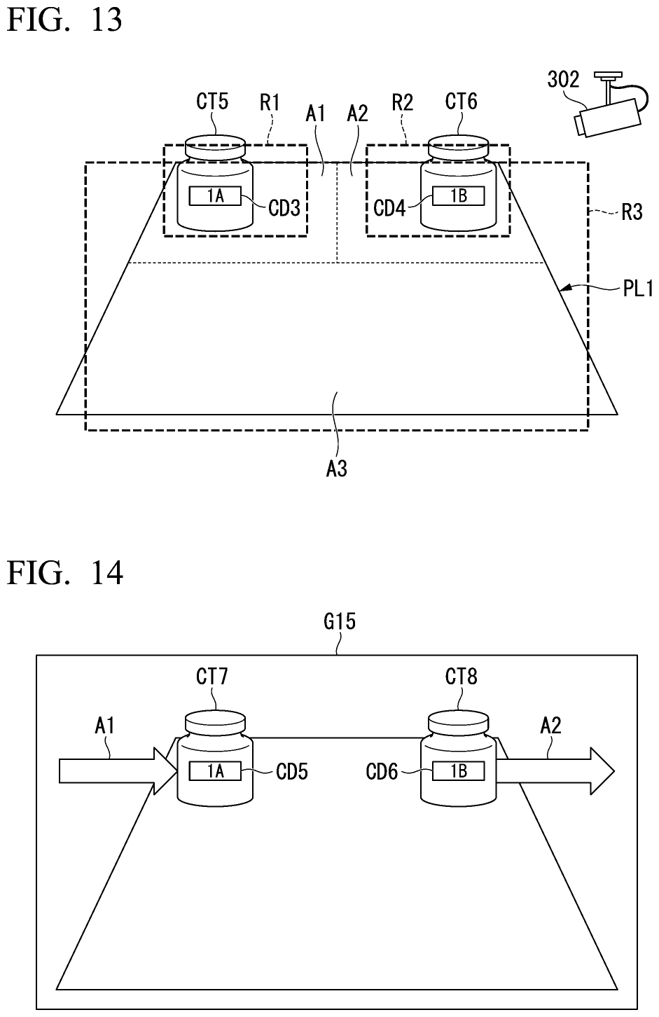

FIG. 13 schematically shows a site where processing on an object is performed. A place PL1 where the processing on the object is performed includes an area A1, an area A2, and an area A3. The area A1 is a region in which a container CT5 storing the object is disposed before the processing is started. The area A2 is a region in which a container CT6 storing the object is disposed after the processing is completed. The area A3 is a region in which the object is disposed and processing on the object is performed. A code CD3 corresponding to first ID information 1A is attached to the container CT5. A code CD4 corresponding to second ID information 1B is attached to the container CT6. The area A1, the area A2, and the area A3 are predetermined.

The camera 302 is disposed such that the area A1, the area A2, and the area A3 are included in a photographing range. In image information generated by the camera 302, a region R1, a region R2, and a region R3 are set. The region R1 is set such that the code attached to the container disposed in the area A1 can be recognized. That is, in an image when the container CT5 is disposed in the area A1, the code CD3 is located in the region RE The region R2 is set such that the code attached to the container disposed in the area A2 can be recognized. That is, in an image when the container CT6 is disposed in the area A2, the code CD4 is located in the region R2. The region R3 is set such that a state of processing in the area A3 can be recognized.

The region R1 corresponds to a first region. The image processing unit 303 acquires first ID information by processing image information of the region R1. The region R2 corresponds to a second region. The image processing unit 303 acquires second ID information by processing image information of the region R2.

The information recording system 10d may include a notification unit. For example, the notification unit instructs the user to dispose the container CT5 in the area A1 before the processing on the object is started. After the processing on the object is completed, the notification unit instructs the user to dispose the container CT6 in the area A2.

The information generation unit 20d may include the image processing unit 303. The image processing unit 303 may generate processing information by extracting the image information of the region R3 from the image information generated by the camera 302.

A third example in which first ID information and second ID information are generated will be described. When a first motion of the ID information is recognized in image information, the image processing unit 303 acquires the ID information as the first ID information. When a second motion of the ID information is recognized in the image information, the image processing unit 303 acquires the ID information as the second ID information. The second motion is different from the first motion.

FIG. 14 shows image information G15 generated by the camera 302. The image information G15 indicates the entire image of a place where processing on an object is performed. The object is stored in a container CT7 before the processing on the object is performed. A code CD5 corresponding to first ID information 1A is attached to the container CT7. After the processing on the object is performed, the object is stored in a container CT8. A code CD6 corresponding to second ID information 1B is attached to the container CT8.

The image processing unit 303 recognizes the code CD5 and the code CD6 from the image information G15. The image processing unit 303 recognizes motions of the code CD5 and the code CD6 on the basis of a plurality of pieces of image information including the image information G15. For example, the image processing unit 303 calculates motion vectors of the code CD5 and the code CD6. As indicated by an arrow A1, the container CT7 to which the code CD5 is attached is moved from the outside of the photographing range of the camera 302 to the inside of the photographing range. When the motion in which the code CD5 is moved from the outside of the photographing range of the camera 302 to the inside of the photographing range is recognized, the image processing unit 303 acquires the code CD5 as the first ID information. As indicated by an arrow A2, the container CT8 to which the code CD6 is attached is moved from the inside of the photographing range of the camera 302 to the outside of the photographing range. When the motion in which the code CD6 is moved from the inside of the photographing range of the camera 302 to the outside of the photographing range is recognized, the image processing unit 303 acquires the code CD6 as the second ID information.

The first motion may be a motion in a first direction and the second motion may be a motion in a second direction different from the first direction. When the motion of the ID information in the first direction is recognized in the image information, the image processing unit 303 may acquire the ID information as the first ID information. When the motion of the ID information in the second direction is recognized in the image information, the image processing unit 303 may acquire the ID information as the second ID information.

As described above, the information generation unit 20d and the ID acquisition unit 30c include the camera 302. Thereby, the information recording system 10d can be easily configured.

### Fifth Modified Example of First Embodiment

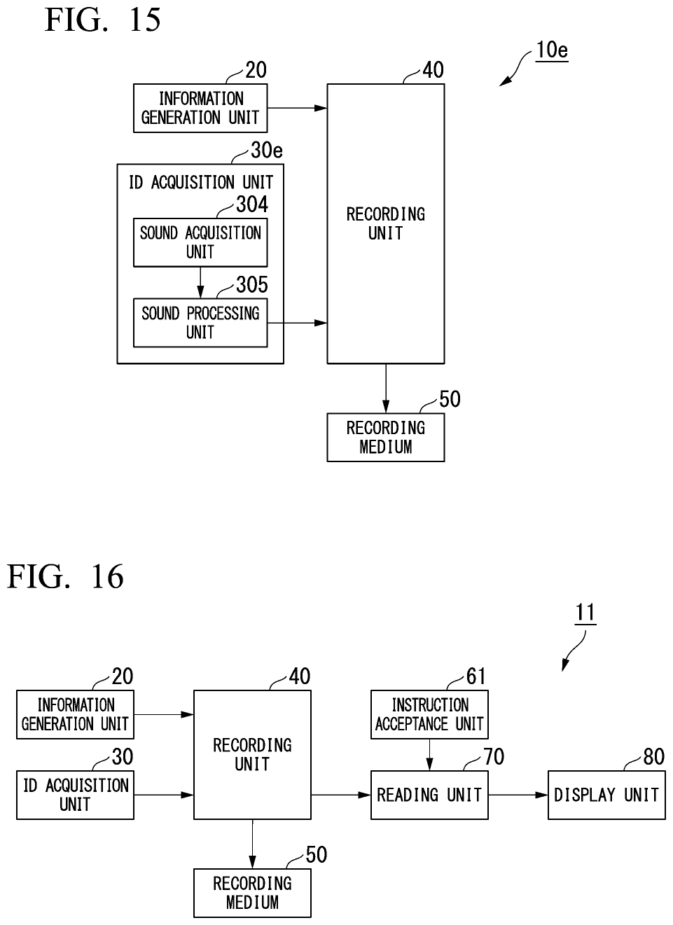

FIG. 15 shows a configuration of an information recording system 10e according to a fifth modified example of the first embodiment of the present invention. Differences from the configuration shown in FIG. 1 will be described in terms of the configuration shown in FIG. 15.

In the information recording system 10e, the ID acquisition unit 30 shown in FIG. 1 is changed to an ID acquisition unit 30e. The ID acquisition unit 30e includes a sound acquisition unit 304 and a sound processing unit 305. The sound acquisition unit 304 acquires sound information based on sound (speech) uttered by the user performing the processing. The sound processing unit 305 recognizes ID information on the basis of the sound information acquired by the sound acquisition unit 304. The sound processing unit 305 may include one or more processors. The sound processing unit 305 may include an ASIC or an FPGA.

For example, the sound acquisition unit 304 is a microphone. The sound acquisition unit 304 may be a wearable microphone worn by the user. The wearable microphone is worn near an observer's mouth. For example, the sound processing unit 305 includes a sound processing circuit configured to perform sound processing.

Except for the points described above, the configuration shown in FIG. 15 is similar to the configuration shown in FIG. 1.

As described above, the ID acquisition unit 30e includes the sound acquisition unit 304 and the sound processing unit 305. Thereby, the information recording system 10e can acquire an ID based on the sound of the user.

### Second Embodiment

FIG. 16 shows a configuration of an information recording system 11 according to a second embodiment of the present invention. Differences from the configuration shown in FIG. 1 will be described in terms of the configuration shown in FIG. 16.

The information recording system 11 has an instruction acceptance unit 61 (a second instruction acceptance unit), a reading unit 70, and a display unit 80 in addition to the configuration of the information recording system 10 shown in FIG. 1. The instruction acceptance unit 61 accepts an instruction for specifying at least one piece of first ID information and second ID information. The reading unit 70 reads processing information associated with at least one piece of the first ID information and the second ID information corresponding to the instruction, and the first ID information and the second ID information associated with the processing information from a recording medium 50. The display unit 80 displays the processing information, the first ID information, and the second ID information read by the reading unit 70.

For example, the instruction acceptance unit 61 is configured as an operation unit. For example, the operation unit includes at least one of a code scanner, an RFID reader, a keyboard, a mouse, a touch pad, a trackball, and a touch panel. The instruction acceptance unit 61 may be configured as a wireless communication unit configured to perform wireless communication with the operation unit.

For example, the reading unit 70 includes a reading processing circuit configured to perform an information reading process. At least one piece of the processing information, the first ID information, and the second ID information recorded on the recording medium 50 may be compressed. Therefore, the reading unit 70 may include a decompression processing circuit configured to decompress the compressed information. The reading unit 70 may include a buffer for a reading process and a decompression process. The reading unit 70 may include one or more processors. The reading unit 70 may include an ASIC or an FPGA.

The display unit 80 is a display device such as a liquid crystal display. For example, the display unit 80 is a monitor of a PC. The display unit 80 may be a wearable display. When the processing information is image information, the display unit 80 may display a thumbnail obtained by reducing image information of one frame. The display unit 80 may display a thumbnail in which at least one piece of the first ID information and the second ID information is recorded.

Except for the points described above, the configuration shown in FIG. 16 is similar to the configuration shown in FIG. 1.

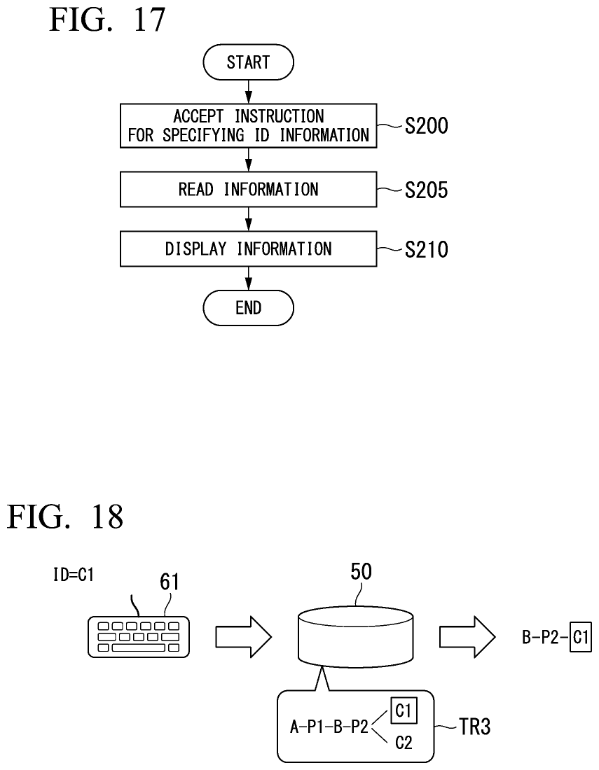

The information recording system 11 performs the process shown in FIG. 2. After the process shown in FIG. 2 is completed, the information recording system 11 performs a process shown in FIG. 17. FIG. 17 shows order of the process to be performed by the information recording system 11. The order of the process to be performed by the information recording system 11 will be described with reference to FIG. 17.

The instruction acceptance unit 61 accepts an instruction for specifying at least one piece of first ID information and second ID information (step S200 (instruction acceptance step)). After step S200, the reading unit 70 reads processing information associated with at least one piece of the first ID information and the second ID information corresponding to the instruction accepted by the instruction acceptance unit 61 from the recording medium 50. Also, the reading unit 70 reads the first ID information and the second ID information associated with the processing information from the recording medium 50 (step S205 (reading step)). After step S205, the display unit 80 displays the processing information, the first ID information, and the second ID information read by the reading unit 70 (step S210 (display step)).

A first example in which information is read from the recording medium 50 will be described. FIG. 18 shows an example of information to be read by the information recording system 11. An example of a process to be performed by the information recording system 11 will be described with reference to FIG. 18.

Information shown in a tree TR3 is recorded on the recording medium 50. Processing information P1, first ID information A, and second ID information B are recorded on the recording medium 50. Processing information P2, first ID information B, second ID information C1, and second ID information C2 are recorded on the recording medium 50.

For example, the instruction acceptance unit 61 accepts an instruction for specifying the second ID information C1. The reading unit 70 reads the processing information P2 associated with the second ID information C1 corresponding to the instruction from the recording medium 50. Also, the reading unit 70 reads the first ID information B and the second ID information C1 associated with the processing information P2 from the recording medium 50.

When the instruction acceptance unit 61 accepts an instruction for specifying the ID information B as the second ID information, the reading unit 70 reads the processing information P1 associated with the second ID information B corresponding to the instruction from the recording medium 50. Also, the reading unit 70 reads the first ID information A and the second ID information B associated with the processing information P1 from the recording medium 50.

When the instruction acceptance unit 61 accepts an instruction for specifying the ID information B as the first ID information, the reading unit 70 reads the processing information P2 associated with the first ID information B corresponding to the instruction from the recording medium 50. Also, the reading unit 70 reads the first ID information B, the second ID information C1, and the second ID information C2 associated with the processing information P2 from the recording medium 50.

The ID information B is used as first ID information and second ID information. An instruction for specifying whether the ID information B is the first ID information or the second ID information may not be accepted. When the ID information B is specified, the reading unit 70 may read information that is to be read when the ID information B is specified as the first ID information and information that is to be read when the ID information B is specified as the second ID information from the recording medium 50.

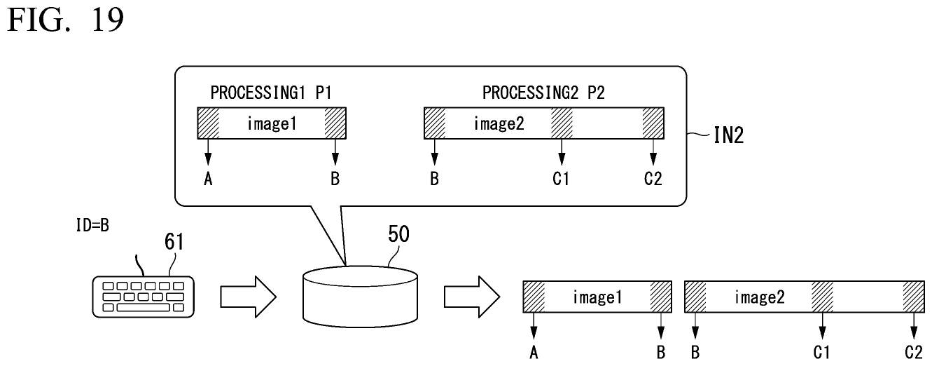

A second example in which information is read from the recording medium 50 will be described. FIG. 19 shows an example of information to be read by the information recording system 11. An example of a process to be performed by the information recording system 11 will be described with reference to FIG. 19.

Information IN2 is recorded on the recording medium 50. The information IN2 includes processing information P1 corresponding to processing 1. The information IN2 includes first ID information A and second ID information B associated with the processing information P1. The processing information P1 includes image information image1. The first ID information A and the second ID information B are added to the image information image1. Also, the information IN2 includes processing information P2 corresponding to processing 2. The information IN2 includes first ID information B, second ID information C1, and second ID information C2 associated with the processing information P2. The processing information P2 includes image information image2. The first ID information B, the second ID information C1, and the second ID information C2 are added to the image information image2.

For example, the instruction acceptance unit 61 accepts an instruction for specifying ID information B. The ID information B is recorded on the recording medium 50 as the second ID information B associated with the image information image1 (the processing information P1). The reading unit 70 reads the image information image1 associated with the second ID information B corresponding to the instruction from the recording medium 50. Also, the reading unit 70 reads the first ID information A and the second ID information B associated with the image information image1 from the recording medium 50. The ID information B is recorded as first ID information B associated with image information image2 (the processing information P2) on the recording medium 50. The reading unit 70 reads the image information image2 associated with the ID information B corresponding to the instruction from the recording medium 50. Also, the reading unit 70 reads the first ID information B, the second ID information C1, and the second ID information C2 associated with the image information image2 from the recording medium 50.

When the information generation unit 20 is a camera and the camera generates moving-image information, the recording unit 40 may record the moving-image information as processing information on the recording medium 50 in parallel with the generation of the moving-image information by the camera. When the processing is performed on the object, the instruction acceptance unit 61 may accept an instruction for specifying the first ID information. At this time, the reading unit 70 reads the processing information recorded on the recording medium 50 by a timing at which the instruction is accepted by the instruction acceptance unit 61 from the recording medium 50. The processing information is moving-image information generated from a timing at which the processing is started to a timing at which the instruction is accepted by the instruction acceptance unit 61. The reading unit 70 may read the processing information recorded on the recording medium 50 at the timing at which the instruction is accepted by the instruction acceptance unit 61 from the recording medium 50. The processing information is image information of one frame generated at the timing at which the instruction is accepted by the instruction acceptance unit 61. A user can check processing details immediately before the timing at which the instruction is accepted by the instruction acceptance unit 61.

A third example in which information is read from the recording medium 50 will be described. When the instruction acceptance unit 61 accepts an instruction for specifying second ID information, the reading unit 70 reads first processing information associated with the second ID information corresponding to the instruction from the recording medium 50. Also, the reading unit 70 reads first ID information and the second ID information associated with the first processing information from the recording medium 50. The reading unit 70 further reads second processing information associated with the second ID information identical to the first ID information associated with the first processing information from the recording medium 50. Also, the reading unit 70 reads the first ID information and the second ID information associated with the second processing information from the recording medium 50.

The reading unit 70 iterates the above-described process until the second ID information identical to the first ID information is not found. When a plurality of pieces of first ID information are associated with one piece of processing information, the reading unit 70 may read each of the plurality of pieces of first ID information from the recording medium 50. For example, when a new object is generated by combining a plurality of objects, the plurality of pieces of first ID information are associated with the processing information. The reading unit 70 reads second processing information associated with the second ID information identical to each of the plurality of pieces of first ID information from the recording medium 50.

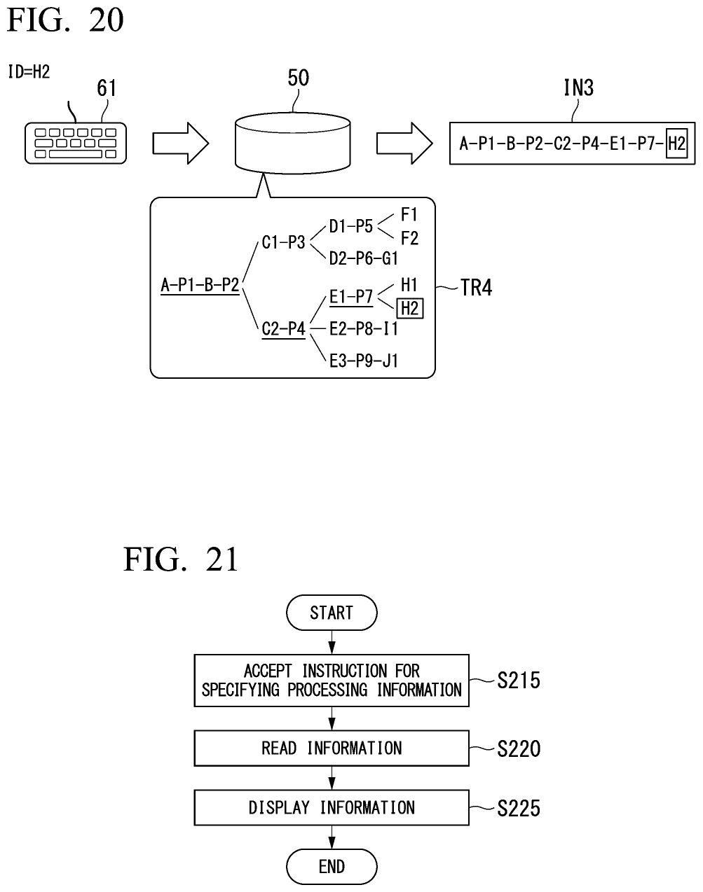

FIG. 20 shows an example of information to be read by the information recording system 11. An example of a process to be performed by the information recording system 11 will be described with reference to FIG. 20.

Information shown in a tree TR4 is recorded on the recording medium 50. Processing information P1 is recorded on the recording medium 50. First ID information A and second ID information B associated with the processing information P1 are recorded on the recording medium 50. Likewise, processing information P2, first ID information B, second ID information C1, and second ID information C2 are recorded on the recording medium 50. Processing information P3, first ID information C1, second ID information D1, and second ID information D2 are recorded on the recording medium 50. Processing information P4, first ID information C2, second ID information E1, second ID information E2, and second ID information E3 are recorded on the recording medium 50. Processing information P5, first ID information D1, second ID information F1, and second ID information F2 are recorded on the recording medium 50. Processing information P6, first ID information D2, and second ID information G1 are recorded on the recording medium 50. Processing information P7, first ID information E1, second ID information H1, and second ID information H2 are recorded on the recording medium 50. Processing information P8, first ID information E2, and second ID information I1 are recorded on the recording medium 50. Processing information P9, first ID information E3, and second ID information J1 are recorded on the recording medium 50.

For example, the instruction acceptance unit 61 accepts an instruction for specifying the second ID information H2. The reading unit 70 reads the processing information P7 associated with the second ID information H2 corresponding to the instruction from the recording medium 50. Also, the reading unit 70 reads the first ID information E1 and the second ID information H2 associated with the processing information P7 from the recording medium 50. Further, the reading unit 70 reads the processing information P4 associated with the second ID information E1 identical to the first ID information E1 associated with the processing information P7 from the recording medium 50. Also, the reading unit 70 reads the first ID information C2 and the second ID information E1 associated with the processing information P4 from the recording medium 50.

The reading unit 70 iterates a process similar to the above-described process. Thereby, the reading unit 70 reads the processing information P2, the first ID information B, and the second ID information C2 from the recording medium 50. Also, the reading unit 70 reads the processing information P1, the first ID information A, and the second ID information B from the recording medium 50. By performing the above-described process, the reading unit 70 reads information IN3 from the recording medium 50.

In this manner, the reading unit 70 can read information indicating which processing process the object has been obtained through on the basis of the instruction for specifying the second ID information.

The information recording system 11 may perform the following processing. When the instruction acceptance unit 61 accepts an instruction for specifying the first ID information, the reading unit 70 reads the first processing information associated with the first ID information corresponding to the instruction from the recording medium 50. Also, the reading unit 70 reads the first ID information and the second ID information associated with the first processing information from the recording medium 50. The reading unit 70 further reads second processing information associated with the first ID information identical to the second ID information associated with the first processing information from the recording medium 50. Also, the reading unit 70 reads the first ID information and the second ID information associated with the second processing information from the recording medium 50.

The reading unit 70 iterates the above-described process until the first ID information identical to the second ID information is not found. When a plurality of pieces of second ID information are associated with one piece of processing information, the reading unit 70 may read each of the plurality of pieces of second ID information from the recording medium 50. For example, when the object is divided by performing processing on the object, the plurality of pieces of second ID information are associated with the processing information. The reading unit 70 reads second processing information associated with first ID information identical to each of the plurality of second ID information from the recording medium 50.

For example, in the example shown in FIG. 20, the instruction acceptance unit 61 accepts an instruction for specifying the first ID information C1. The reading unit 70 reads the processing information P3 associated with the first ID information C1 corresponding to the instruction from the recording medium 50. Also, the reading unit 70 reads the first ID information C1, the second ID information D1, and the second ID information D2 associated with the processing information P3 from the recording medium 50. Further, the reading unit 70 reads the processing information P5 associated with the first ID information D1 identical to the second ID information D1 associated with the processing information P3 from the recording medium 50. Also, the reading unit 70 reads the first ID information D1, the second ID information F1, and the second ID information F2 associated with the processing information P5 from the recording medium 50. Further, the reading unit 70 reads the processing information P6 associated with the first ID information D2 identical to the second ID information D2 associated with the processing information P3 from the recording medium 50. Also, the reading unit 70 reads the first ID information D2 and the second ID information G1 associated with the processing information P6 from the recording medium 50.

In this manner, the reading unit 70 can read information indicating which object is finally obtained through which processing process the object has gone on the basis of the instruction for specifying the first ID information.

As described above, the information recording system 11 includes the instruction acceptance unit 61 and the reading unit 70. Thereby, the information recording system 11 can acquire information associated with the ID information corresponding to the instruction accepted by the instruction acceptance unit 61 from the recording medium 50.

As described above, the information recording system 11 includes the display unit 80. Thereby, the information recording system 11 can display the information acquired from the recording medium 50. When a plurality of types of processing are performed on the object, the user can verify the validity of the processing process on the basis of the processing information corresponding to each type of processing. Also, the user can ascertain a relationship between the plurality of types of processing on the basis of the ID information corresponding to each type of processing.

As described above, the reading unit 70 reads information related to a plurality of types of processing sequentially performed on the object from the recording medium 50. Thereby, the information recording system 11 can acquire a history of processing on the object of interest.

### Third Embodiment

A third embodiment of the present invention will be described using an information recording system 11 shown in FIG. 16. In the third embodiment, an instruction acceptance unit 61 (a third instruction acceptance unit) accepts an instruction for specifying processing information. A reading unit 70 reads the processing information corresponding to the instruction and first ID information and second ID information associated with the processing information from a recording medium 50. A display unit 80 displays the processing information, the first ID information, and the second ID information read by the reading unit 70.

The information recording system 11 performs the process shown in FIG. 2. After the process shown in FIG. 2 is completed, the information recording system 11 performs a process shown in FIG. 21. FIG. 21 shows order of the process to be performed by the information recording system 11. The order of the process to be performed by the information recording system 11 will be described with reference to FIG. 21.

The instruction acceptance unit 61 accepts an instruction for specifying processing information (step S215 (instruction acceptance step)). After step S215, the reading unit 70 reads the processing information corresponding to the instruction accepted by the instruction acceptance unit 61 from the recording medium 50. Also, the reading unit 70 reads first ID information and second ID information associated with the processing information from the recording medium 50 (step S220 (reading step)). After step S220, the display unit 80 displays the processing information, the first ID information, and the second ID information read by the reading unit 70 (step S225 (display step)).

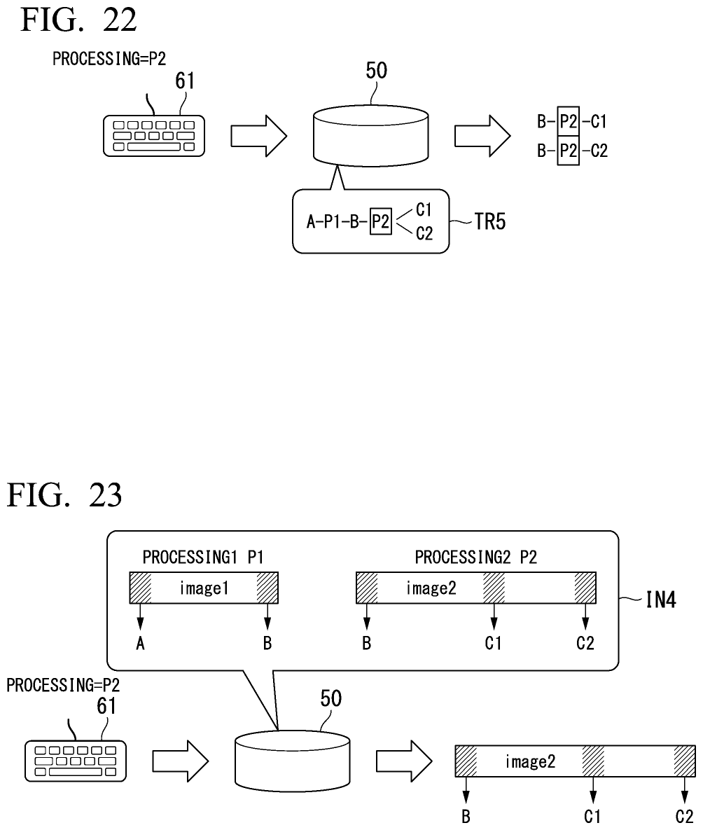

A first example in which information is read from the recording medium 50 will be described. FIG. 22 shows an example of information to be read by the information recording system 11. An example of a process to be performed by the information recording system 11 will be described with reference to FIG. 22.

Information shown in a tree TR5 is recorded on the recording medium 50. The information shown in the tree TR5 is identical to the information shown in the tree TR3 shown in FIG. 18.

For example, the instruction acceptance unit 61 accepts an instruction for specifying processing information P2. The reading unit 70 reads the processing information P2 corresponding to the instruction from the recording medium 50. Also, the reading unit 70 reads first ID information B, second ID information C1, and second ID information C2 associated with the processing information P2 from the recording medium 50. As described above, when a plurality of pieces of second ID information are associated with the processing information corresponding to the instruction accepted by the instruction acceptance unit 61, the reading unit 70 reads each of a plurality of pieces of second ID information from the recording medium 50.

A second example in which information is read from the recording medium 50 will be described. FIG. 23 shows an example of information to be read by the information recording system 11. An example of a process to be performed by the information recording system 11 will be described with reference to FIG. 23.

Information IN4 is recorded on the recording medium 50. The information IN4 is identical to the information IN2 in FIG. 19.

For example, the instruction acceptance unit 61 accepts an instruction for specifying the processing information P2. The processing information P2 is recorded as image information image2 on the recording medium 50. The reading unit 70 reads the image information image2 corresponding to the instruction from the recording medium 50. Also, the reading unit 70 reads first ID information B, second ID information C1, and second ID information C2 associated with the image information image2 from the recording medium 50.

A third example in which information is read from the recording medium 50 will be described. When the instruction acceptance unit 61 accepts an instruction for specifying first processing information, the reading unit 70 reads the first processing information corresponding to the instruction from the recording medium 50. Also, the reading unit 70 reads first ID information and second ID information associated with the first processing information from the recording medium 50. Further, the reading unit 70 reads second processing information associated with second ID information identical to the first ID information associated with the first processing information from the recording medium 50. Also, the reading unit 70 reads first ID information and the second ID information associated with the second processing information from the recording medium 50.

The reading unit 70 iterates the above-described process until the second ID information identical to the first ID information is not found. When a plurality of pieces of first ID information are associated with one piece of processing information, the reading unit 70 may read each of the plurality of pieces of first ID information from the recording medium 50. The reading unit 70 reads the second processing information associated with the second ID information identical to each of the plurality of pieces of first ID information from the recording medium 50.

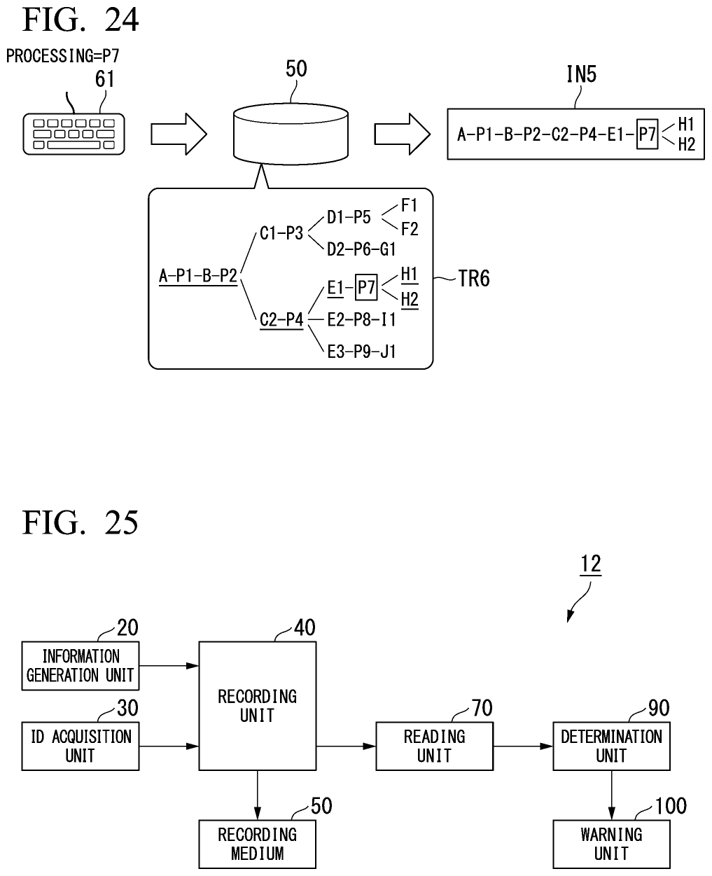

FIG. 24 shows an example of information to be read by the information recording system 11. An example of a process to be performed by the information recording system 11 will be described with reference to FIG. 24.

Information shown in a tree TR6 is recorded on the recording medium 50. The information shown in the tree TR6 is identical to the information shown in the tree TR4 shown in FIG. 20.

For example, the instruction acceptance unit 61 accepts an instruction for specifying processing information P7. The reading unit 70 reads the processing information P7 corresponding to the instruction from the recording medium 50. Also, the reading unit 70 reads first ID information E1, second ID information H1, and second ID information H2 associated with the processing information P7 from the recording medium 50. Further, the reading unit 70 reads processing information P4 associated with second ID information E1 identical to the first ID information E1 associated with the processing information P7 from the recording medium 50. Also, the reading unit 70 reads first ID information C2 and the second ID information E1 associated with the processing information P4 from the recording medium 50.

The reading unit 70 iterates a process similar to the above-described process. Thereby, the reading unit 70 reads processing information P2, first ID information B, and second ID information C2 from the recording medium 50. Also, the reading unit 70 reads processing information P1, first ID information A, and second ID information B from the recording medium 50. By performing the above-described process, the reading unit 70 reads information IN5 from the recording medium 50.

The information recording system 11 may perform the following process. When the instruction acceptance unit 61 accepts an instruction for specifying first processing information, the reading unit 70 reads the first processing information corresponding to the instruction from the recording medium 50. Also, the reading unit 70 reads first ID information and second ID information associated with the first processing information from the recording medium 50. Further, the reading unit 70 reads second processing information associated with first ID information identical to the second ID information associated with the first processing information from the recording medium 50. Also, the reading unit 70 reads the first ID information and second ID information associated with the second processing information from the recording medium 50.

The reading unit 70 iterates the above-described process until the first ID information identical to the second ID information is not found. When a plurality of pieces of second ID information are associated with one piece of processing information, the reading unit 70 may read each of the plurality of pieces of second ID information from the recording medium 50. The reading unit 70 reads second processing information associated with the first ID information identical to each of the plurality of pieces of second ID information from the recording medium 50.

For example, in the example shown in FIG. 24, the instruction acceptance unit 61 accepts an instruction for specifying processing information P3. The reading unit 70 reads the processing information P3 corresponding to the instruction from the recording medium 50. Also, the reading unit 70 reads first ID information C1, second ID information D1, and second ID information D2 associated with the processing information P3 from the recording medium 50. Further, the reading unit 70 reads processing information P5 associated with first ID information D1 identical to the second ID information D1 associated with the processing information P3 from the recording medium 50. Also, the reading unit 70 reads the first ID information D1, second ID information F1, and second ID information F2 associated with the processing information P5 from the recording medium 50. Further, the reading unit 70 reads processing information P6 associated with first ID information D2 identical to the second ID information D2 associated with the processing information P3 from the recording medium 50. Also, the reading unit 70 reads the first ID information D2 and second ID information G1 associated with the processing information P6 from the recording medium 50.

As described above, the information recording system 11 includes the instruction acceptance unit 61 and the reading unit 70. Thereby, the information recording system 11 can acquire information associated with processing information corresponding to an instruction from the recording medium 50.

As described above, the information recording system 11 includes the display unit 80. Thus, the information recording system 11 can display information acquired from the recording medium 50. When a plurality of types of processing are performed on an object, a user can verify the validity of processing process on the basis of processing information corresponding to each type of processing. Also, the user can ascertain a relationship between the plurality of types of processing on the basis of ID information corresponding to each type of processing.

As described above, the reading unit 70 reads information about a plurality of types of processing to be sequentially performed on an object from the recording medium 50. Thereby, the information recording system 11 can acquire a history of a series of types of processing including processing of interest.

### Fourth Embodiment

FIG. 25 shows a configuration of an information recording system 12 according to a fourth embodiment of the present invention. Differences from the configuration shown in FIG. 1 will be described in terms of the configuration shown in FIG. 25.

The information recording system 12 includes a reading unit 70, a determination unit 90, and a warning unit 100 in addition to the configuration of the information recording system 10 shown in FIG. 1.

The reading unit 70 reads at least one piece of processing information, first ID information, and second ID information recorded on a recording medium 50. The determination unit 90 determines whether or not at least one piece of the processing information, the first ID information, and the second ID information recorded on the recording medium 50 matches a condition predefined as a condition to be satisfied by the processing information, the first ID information, and the second ID information. The warning unit 100 issues a warning when at least one piece of the processing information, the first ID information, and the second ID information recorded on the recording medium 50 does not match the above-described condition.

For example, the determination unit 90 includes an information processing circuit configured to perform information processing. The determination unit 90 may include one or more processors. The determination unit 90 may include an ASIC or an FPGA.

For example, the warning unit 100 is a display unit such as a liquid crystal display. The warning unit 100 may be a speaker. For example, condition information indicating the condition to be satisfied by at least one piece of the processing information, the first ID information, and the second ID information is pre-recorded on the recording medium 50. The reading unit 70 reads at least one piece of the processing information, the first ID information, and the second ID information corresponding to the processing from the recording medium 50 at a timing at which the processing on the object is completed or the like. Also, the reading unit 70 reads the condition information corresponding to the processing from the recording medium 50. The determination unit 90 compares at least one piece of the processing information, the first ID information, and the second ID information with the condition indicated by the condition information. When at least one piece of the processing information, the first ID information, and the second ID information does not match the condition indicated by the condition information, the warning unit 100 issues a warning. A process to be performed by the determination unit 90 may include a process of comparing a state of at least one piece of the processing information, the first ID information, and the second ID information with the condition indicated by the condition information.

For example, when two pieces of first ID information are recorded in processing in which two objects are generated by dividing one object, the warning unit 100 issues a warning. Alternatively, when only one piece of second ID information or three or more pieces of second ID information are recorded in the processing, the warning unit 100 issues a warning. When the information generation unit 20 includes a sensor mounted on an instrument used for the processing, processing information corresponding to the used instrument is recorded. When the recorded processing information is different from the processing information indicated by the condition information, i.e., when an instrument different from the instrument to be used is used, the warning unit 100 issues a warning. When a container used in the processing is predefined and ID information is attached to the container, the ID information is included in the condition information. When the recorded ID information is different from the ID information indicated by the condition information, i.e., when a container different from the container to be used is used, the warning unit 100 issues a warning.

Except for the points described above, the configuration shown in FIG. 25 is similar to the configuration shown in FIG. 1.

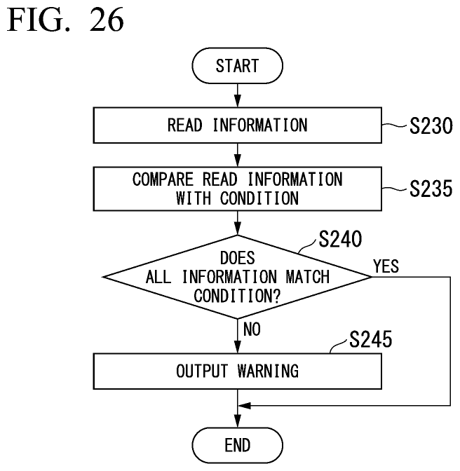

The information recording system 12 performs the process shown in FIG. 2. After the process shown in FIG. 2 is completed, the information recording system 12 performs a process shown in FIG. 26. FIG. 26 shows order of the process to be performed by the information recording system 12. The order of the process to be performed by the information recording system 12 will be described with reference to FIG. 26.

The reading unit 70 reads at least one piece of processing information, first ID information, and second ID information from the recording medium 50. Also, the reading unit 70 reads condition information from the recording medium 50 (step S230 (reading step)). For example, the reading unit 70 reads at least one piece of the processing information, the first ID information, and the second ID information recorded on the recording medium 50 from the recording medium 50 at a timing closest to a current time point. Also, the reading unit 70 reads condition information corresponding to processing specified by a user from the recording medium 50.

After step S230, the determination unit 90 compares at least one piece of the processing information, the first ID information, and the second ID information read from the recording medium 50 with a condition indicated by the condition information (step S235 (comparison step)). After step S235, the determination unit 90 determines whether or not all the information read from the recording medium 50 matches the condition indicated by the condition information (step S240 (determination step)).

In step S240, when all the information read from the recording medium 50 matches the condition indicated by the condition information, no warning is output. When at least one piece of the information read from the recording medium 50 does not match the condition indicated by the condition information in step S240, the warning unit 100 outputs a warning (step S245 (warning step)).

The information recording system 12 may include the instruction acceptance unit 61 and the display unit 80 shown in FIG. 16.

As described above, the information recording system 12 includes the determination unit 90 and the warning unit 100. Thereby, the information recording system 12 can reduce the number of operation errors that will occur. When an operation error occurs, the information recording system 12 can support the verification of the validity of a processing situation as in the information recording system 10 of the first embodiment.

### Fifth Embodiment

A fifth embodiment of the present invention will be described using the information recording system 11 shown in FIG. 16. In the fifth embodiment, an example in which the information recording system 11 is applied to pathological diagnosis will be described.

Pathological specimens in the pathological diagnosis are made in the following procedure.

### (1) Reception

An object such as a patient's organ arrives at an inspection room. At a reception, detailed information of the object is input to a computer.

### (2) Cutting

Parts necessary for diagnosis are cut from the object.

### (3) Embedding

The cut object is placed inside a processor (an embedding device) and processed. Thereby, water in tissue is converted into paraffin. The object is embedded in the paraffin.

### (4) Slicing

The object solidified by paraffin is sliced thinly by a slicing machine. Flakes of the object are attached to a glass slide.

### (5) Staining

The object is stained by a staining machine for observation using a microscope. For example, staining such as Hematoxylin-Eosin (HE) staining and immunostaining is performed. After the staining is performed, a cover glass is attached.

Pathological specimens are made by the above-described procedure. The pathologist observes the pathological specimens and performs diagnosis.

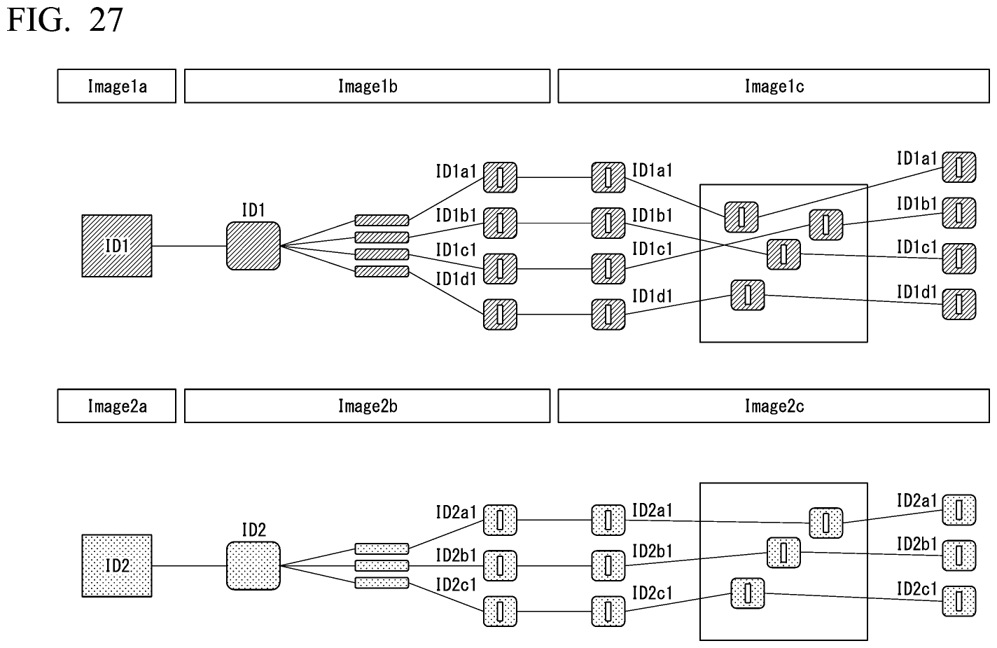

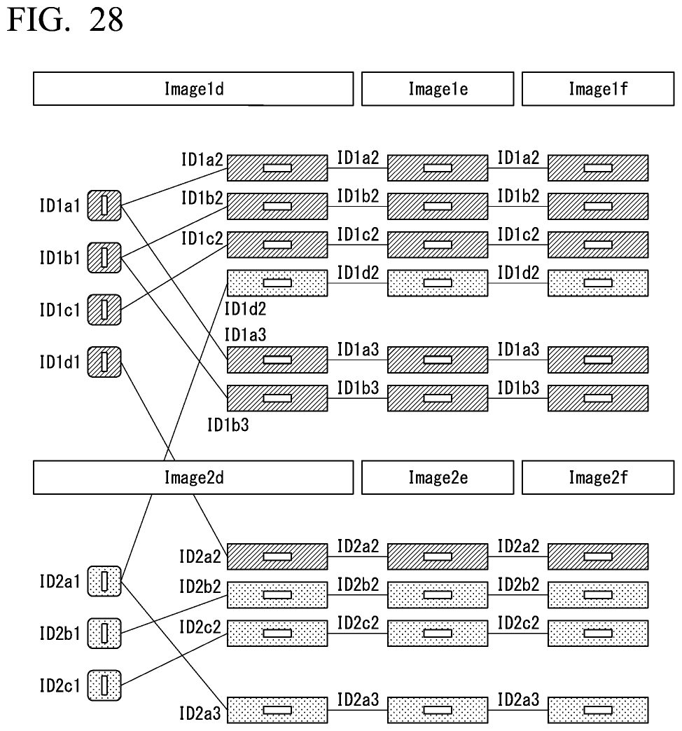

FIGS. 27 and 28 show a first example of pathological diagnosis. A method of checking a cause of the mix-up of objects in the first example of the pathological diagnosis will be described with reference to FIGS. 27 and 28.

A first container storing an object arrives at the reception. ID information ID1 is attached to the first container. A user A performs necessary processing at the reception. A state of processing is photographed by a camera and image information Image1a that is processing information is generated. The image information Image1a, first ID information ID1, and second ID information ID1 are recorded on a recording medium 50.

Thereafter, a second container storing another object arrives at the reception. ID information ID2 is attached to the second container. The user A performs necessary processing at the reception. A state of processing is photographed by the camera and image information Image2a that is processing information is generated. The image information Image2a, first ID information ID2, and second ID information ID2 are recorded on the recording medium 50. The first container and the second container storing the object are moved from the reception to a place where cutting is performed.

A user B takes out the object from the first container and cuts necessary parts from the object. As shown in FIG. 27, four parts are cut from the object. The cut parts are put into cartridges. Each piece of ID information ID1a1, ID information ID1b1, ID information ID1c1, and ID information ID1d1 is attached to one cartridge. A state of processing is photographed by the camera and image information Image1b that is processing information is generated. The image information Image1b, first ID information ID1, and second ID information are recorded on the recording medium 50. As the second ID information, the ID information ID1a1, the ID information ID1b1, the ID information ID1c1, and the ID information ID1d1 are recorded on the recording medium 50.

Thereafter, the user B takes out the object from the second container and cuts necessary parts from the object. As shown in FIG. 27, three parts are cut from the object. The cut parts are put into cartridges. Each piece of ID information ID2a1, ID information ID2b1, and ID information ID2c1 is attached to one cartridge. A state of processing is photographed by the camera and image information Image2b that is processing information is generated. The image information Image2b, first ID information ID2, and second ID information are recorded on the recording medium 50. As the second ID information, the ID information ID2a1, the ID information ID2b1, and the ID information ID2c1 are recorded on the recording medium 50. The cartridge storing the object is moved to a place where embedding is performed.

A user C puts the cartridges into a processor and starts the processor. For example, the processing is simultaneously performed on the four cartridges including the parts of the object stored in the first container. After the processing is complete, the user C removes the cartridge from the processor. A state of processing is photographed by the camera and image information Image1c that is processing information is generated. The image information Image1c, first ID information, and second ID information are recorded on the recording medium 50. The first ID information and the second ID information are identical. The ID information ID1a1, the ID information ID1b1, the ID information ID1c1, and the ID information ID1d1 are recorded as the first ID information and the second ID information on the recording medium 50.

Processing is performed simultaneously on the three cartridges including the parts of the object stored in the second container. After the processing is completed, the user C removes the cartridges from the processor. A state of processing is photographed by the camera and image information Image2c that is processing information is generated. The image information Image2c, first ID information, and second ID information are recorded on the recording medium 50. The first ID information and the second ID information are identical. The ID information ID2a1, the ID information ID2b1, and the ID information ID2c1 are recorded as the first ID information and the second ID information on the recording medium 50. The processing may be performed simultaneously on the seven cartridges described above. After embedding is completed, the cartridges are moved to a place where slicing is performed.

A user D removes the object from the cartridge and slices the object. Two slices are cut from the object taken out of the cartridge to which the ID information ID1a1 is attached. One of the two slices is placed on a glass slide to which ID information ID1a2 is attached. The other of the two slices is placed on a glass slide to which ID information ID1a3 is attached. Two slices are cut from the object taken out of the cartridge to which the ID information ID1b1 is attached. One of the two slices is placed on a glass slide to which ID information ID1b2 is attached. The other of the two slices is placed on a glass slide to which ID information ID1b3 is attached.

One slice is cut from the object taken out of the cartridge to which the ID information ID1c1 is attached. The slice is placed on a glass slide to which ID information ID1c2 is attached. One slice is cut from the object taken out of the cartridge to which the ID information ID1d1 is attached. When the user D stores the slice in the glass slide, the glass slide in which the slice is required to be stored is mistaken. For this reason, the slice is placed on a glass slide to which ID information ID2a2 is attached. ID information ID1d2 is attached to a glass slide in which the slice is required to be originally stored.

A state of processing is photographed by the camera and image information Image1d that is processing information is generated. The image information Image1d, first ID information, and second ID information are recorded on the recording medium 50. In a first combination of the first ID information and the second ID information, the first ID information is the ID information ID1a1 and the second ID information is the ID information ID1a2 and the ID information ID1a3. In a second combination of the first ID information and the second ID information, the first ID information is the ID information ID1b1 and the second ID information is the ID information ID1b2 and the ID information ID1b3. In a third combination of the first ID information and the second ID information, the first ID information is the ID information ID1c1 and the second ID information is the ID information ID1c2. In a fourth combination of the first ID information and the second ID information, the first ID information is the ID information ID1d1 and the second ID information is the ID information ID2a2.

Two slices are cut from the object taken out of the cartridge to which the ID information ID2a1 is attached. When the user D stores one of the two slices in a glass slide, a glass slide in which the slice is required to be stored is mistaken. For this reason, the slice is placed on a glass slide to which the ID information ID1d2 is attached. The ID information ID2a2 is attached to the glass slide in which the slice is required to be originally stored. The other of the two slices is placed on a glass slide to which ID information ID2a3 is attached.

One slice is cut from the object taken out of the cartridge to which the ID information ID2b1 is attached. The slice is placed on a glass slide to which ID information ID2b2 is attached. One slice is cut from the object taken out of a cartridge to which the ID information ID2c1 is attached. The slice is placed on a glass slide to which ID information ID2c2 is attached.

A state of processing is photographed by the camera and image information Image2d that is processing information is generated. The image information Image2d, first ID information, and second ID information are recorded on the recording medium 50. In a first combination of the first ID information and the second ID information, the first ID information is the ID information ID2a1 and the second ID information is the ID information ID1d2 and ID information ID2a3. In a second combination of the first ID information and the second ID information, the first ID information is the ID information ID2b1 and the second ID information is the ID information ID2b2. In a third combination of the first ID information and the second ID information, the first ID information is the ID information ID2c1 and the second ID information is the ID information ID2c2. The glass slides are moved to a place where staining is performed.

A user E performs HE staining or immunostaining on an object placed on a glass slide. For example, the user E performs the HE staining on the object placed on the glass slide to which each piece of the ID information ID1a2, the ID information ID1b2, the ID information ID1c2, and the ID information ID1d2 is attached. Also, the user F performs the immunostaining on the object placed on the glass slide to which each piece of the ID information ID1a3 and the ID information ID1b3 is attached. After the staining is completed, a cover glass is attached to complete pathological specimens.

A state of processing is photographed by the camera and image information Image1e that is processing information is generated. The image information Image1e, first ID information, and second ID information are recorded on the recording medium 50. The first ID information and the second ID information are identical. The ID information ID1a2, the ID information ID1b2, the ID information ID1c2, the ID information ID1d2, the ID information ID1a3, and the ID information ID1b3 are recorded as the first ID information and the second ID information on the recording medium 50.

Thereafter, the user E performs the HE staining on the object placed on the glass slide to which each piece of the ID information ID2a2, the ID information ID2b2, and the ID information ID2c2 is attached. Also, the user F performs the immunostaining on the object placed on a glass slide to which the ID information ID2a3 is attached. After the staining is completed, a cover glass is attached to complete a pathological specimen.

A state of processing is photographed by the camera and image information Image2e that is processing information is generated. The image information Image2e, first ID information, and second ID information are recorded on the recording medium 50. The first ID information and the second ID information are identical. The ID information ID2a2, the ID information ID2b2, the ID information ID2c2, and the ID information ID2a3 are recorded as the first ID information and the second ID information on the recording medium 50. Pathological specimens are moved to a place where diagnosis is performed.

A diagnostician user G observes the pathological specimens with a microscope and performs diagnosis. The user G performs the diagnosis on the basis of the pathological specimen to which each piece of the ID information ID1a2, the ID information ID1b2, the ID information ID1c2, the ID information ID1d2, the ID information ID1a3 and the ID information ID1b3 is attached.

A state of diagnosis is photographed by the camera and image information Image1f that is processing information is generated. The image information Image1f, first ID information, and second ID information are recorded on the recording medium 50. The first ID information and the second ID information are identical. The ID information ID1a2, the ID information ID1b2, the ID information ID1c2, the ID information ID1d2, the ID information ID1a3, and the ID information ID1b3 are recorded as the first ID information and the second ID information on the recording medium 50.

Thereafter, the user G performs the diagnosis on the basis of the pathological specimen to which each piece of the ID information ID2a2, the ID information ID2b2, the ID information ID2c2, and the ID information ID2a3 is attached. A state of diagnosis is photographed by the camera and image information Image 2f that is processing information is generated. The image information Image 2f, first ID information, and second ID information are recorded on the recording medium 50. The first ID information and the second ID information are identical. The ID information ID2a2, the ID information ID2b2, the ID information ID2c2, and the ID information ID2a3 are recorded as the first ID information and the second ID information on the recording medium 50.

The user G notices that the pathological specimen to which the ID information ID2a2 is attached is suspicious. Thus, there is a possibility that the mix-up of objects may have occurred. It is difficult to check all of objects within the cartridges and objects on the glass slides. For example, in the fifth embodiment, the user (verifier) can check the cause of the mix-up by the following method.

The instruction acceptance unit 61 accepts an instruction for specifying the second ID information ID2a2. The reading unit 70 reads the image information Image2f, the first ID information ID2a2, and the second ID information ID2a2 from the recording medium 50. The display unit 80 displays the image information Image2f, the first ID information ID2a2, and the second ID information ID2a2. The verifier checks whether or not the user E and the user F have performed suspicious behavior on the basis of the image information Image2f. When the cause of the mix-up cannot be found from the image information Image2f, the verifier checks image information of processing directly before the current processing.

The reading unit 70 reads the image information Image2e, the first ID information ID2a2, and the second ID information ID2a2 from the recording medium 50. For example, time point information is added to image information. The reading unit 70 can recognize the order in which the image information Image2e and the image information Image2f are generated on the basis of the time point information added to the image information Image2e and the image information Image2f. The display unit 80 displays the image information Image2e, the first ID information ID2a2, and the second ID information ID2a2. The verifier checks whether or not the user E and the user F have performed suspicious behavior on the basis of the image information Image2e. When the cause of the mix-up cannot be found from the image information Image2e, the verifier checks the image information of processing directly before the current processing.

The reading unit 70 reads the image information Image1d, the first ID information ID1d1, and the second ID information ID2a2 from the recording medium 50. The display unit 80 displays the image information Image1d, the first ID information ID1d1, and the second ID information ID2a2. The verifier checks whether or not the user D has performed suspicious behavior on the basis of the image information Image1d. As a result, in the image information Image1d, it is checked that the user D performs an operation different from the slicing operation. For example, there is a possibility that an operation such as taking a telephone call will cause the mix-up. It is checked that the user D has different objects before and after an operation such as a telephone call. It is estimated that the object corresponding to the first ID information ID1d1 has been erroneously placed on a glass slide different from the original. It is necessary to identify an object required to be placed on the glass slide to which the second ID information ID2a2 is attached.

An object processed at a time point close to a time point at which the object corresponding to the ID information ID1d1 was processed is an object to be checked. Thus, the verifier checks the image information Image2d generated at a time point close to a time point at which the image information Image1d was generated during the slicing. As a result, in the image information Image2d, it is checked that the object corresponding to the ID information ID2a1 is placed on the glass slide to which the ID information ID1d2 is attached. Originally, this object is required to be placed on the glass slide to which the ID information ID2a2 is attached.

For example, a relationship between ID information of a container arriving at the reception and ID information attached to a glass slide used for an object stored in the container is predefined. Thus, the verifier can determine that a relationship between the ID information ID2a1 and the ID information ID1d2 is incorrect.

For example, in the image information Image2d, the ID information attached to the container for storing the object can be visually recognized. Thereby, the verifier can ascertain a relationship between the first ID information and the second ID information in the image information Image2d. Image information of each type of processing may be generated during the processing on each of the plurality of objects. The first ID information and the second ID information in each type of processing are associated with the image information in each type of processing. For example, when four objects are processed, four pieces of image information are generated by generating image information independently in each type of processing. First ID information and second ID information are associated with respective image information. The information recording system 12 may include the instruction acceptance unit 60 shown in FIG. 6. The image information Image2d may be divided into a plurality of pieces on the basis of a plurality of start and end instructions accepted by the instruction acceptance unit 60.

The pathological specimen corresponding to the ID information ID1d2 identified as described above is identified as follows. The instruction acceptance unit 61 accepts an instruction for specifying the first ID information ID1d2. The reading unit 70 reads the image information Imagele, the first ID information ID1d2, and the second ID information ID1d2 from the recording medium 50. Furthermore, the reading unit 70 reads the image information Image1f, the first ID information ID1d2, and the second ID information ID1d2 from the recording medium 50. There is a possibility that the pathological specimen to which the ID information ID1d2 is attached may have been mistaken for the pathological specimen to which ID information ID2a2 is attached.

A comparison is made between the pathological specimen to which the ID information ID1c2 is attached and the pathological specimen to which the ID information ID1d2 is attached. These pathological specimens should be cut from the same portion and have common features such as structures. Also, a comparison is made between the pathological specimen to which the ID information ID2a2 is attached and the pathological specimen to which the ID information ID2b2 is attached. These pathological specimens should be cut from the same portion and have common features such as structures. As a result of the above, it is identified that the mix-up of objects has occurred between the pathological specimen to which the ID information ID1d2 is attached and the pathological specimen to which the ID information ID2a2 is attached.

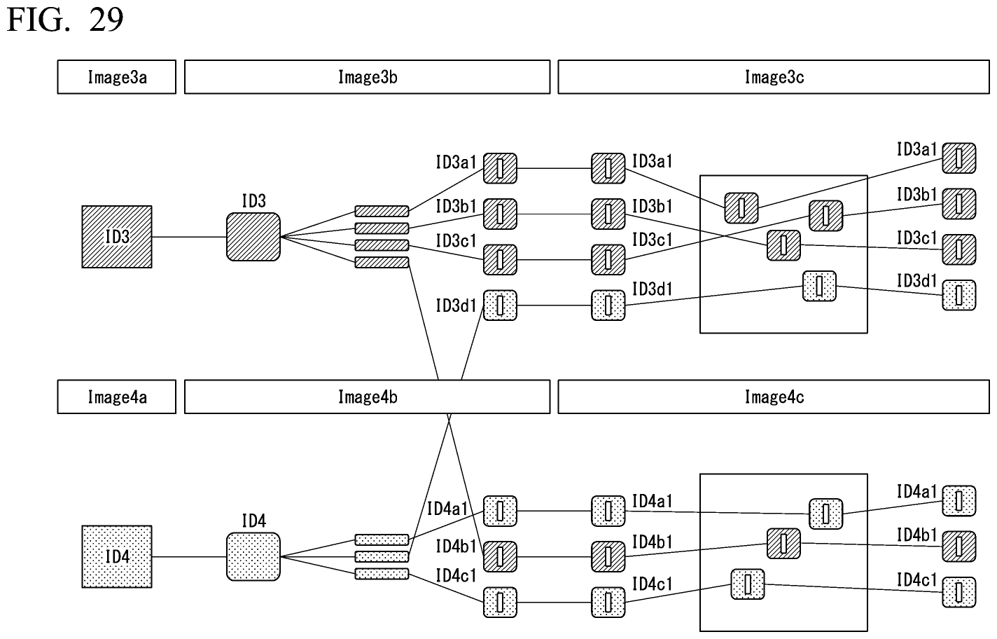

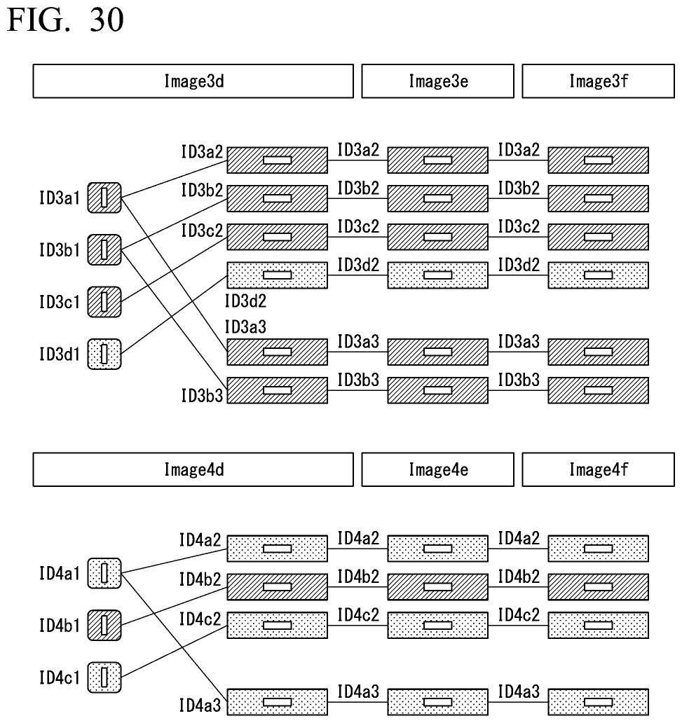

FIGS. 29 and 30 show a second example of pathological diagnosis. A method of checking a cause of the mix-up of objects in the second example of the pathological diagnosis will be described with respect to FIGS. 29 and 30. Description common to the description in the first example will be omitted.

A first container storing an object arrives at the reception. ID information ID3 is attached to the first container. Image information Image3a, first ID information ID3, and second ID information ID3 are recorded on the recording medium 50. Thereafter, a second container storing another object arrives at the reception. ID information ID4 is attached to the second container. Image information Image4a, first ID information ID4, and second ID information ID4 are recorded on the recording medium 50.

Four parts are cut from the object stored in the first container. One of the four parts is put into a cartridge to which ID information ID3a1 is attached. Another part of the four parts is put into a cartridge to which ID information ID3b1 is attached. Another part of the four parts is put into a cartridge to which ID information ID3c1 is attached. When a user B stores the remaining one part in a cartridge, the cartridge in which the part is required to be stored is mistaken. For this reason, the part is put into a cartridge to which ID information ID4b1 is attached. ID information ID3d1 is attached to a glass slide in which the part is required to be originally stored. Image information Image3b, first ID information ID3, and second ID information are recorded on the recording medium 50. As the second ID information, the ID information ID3a1, the ID information ID3b1, the ID information ID3c1, and the ID information ID4b1 are recorded on the recording medium 50.

Thereafter, three parts are cut from an object stored in a second container. One of the three parts is put into a cartridge to which ID information ID4a1 is attached. Another part of the three parts is put into a cartridge to which ID information ID4c1 is attached. When the user B stores the remaining one part in a cartridge, the cartridge in which the part is required to be stored is mistaken. For this reason, the part is put into a cartridge to which the ID information ID3d1 is attached. The ID information ID4b1 is attached to a glass slide in which the part is required to be originally stored. Image information Image4b, first ID information ID4 and second ID information are recorded on the recording medium 50. As the second ID information, the ID information ID4a1, the ID information ID4c1, and the ID information ID3d1 are recorded on the recording medium 50.

In embedding, image information Image3c, first ID information, and second ID information are recorded on the recording medium 50. The first ID information and the second ID information are identical. The ID information ID3a1, the ID information ID3b1, the ID information ID3c1, and the ID information ID3d1 are recorded as the first ID information and the second ID information on the recording medium 50.

Also, in the embedding, image information Image4c, first ID information, and second ID information are recorded on the recording medium 50. The first ID information and the second ID information are identical. The ID information ID4a1, the ID information ID4b1, and the ID information ID4c1 are recorded as the first ID information and the second ID information on the recording medium 50.

By performing a slicing process, two slices are cut from the object taken out of the cartridge to which the ID information ID3a1 is attached. One of the two slices is placed on a glass slide to which ID information ID3a2 is attached. The other of the two slices is placed on a glass slide to which ID information ID3a3 is attached. Two slices are cut from the object taken out of the cartridge to which ID information ID3b1 is attached. One of the two slices is placed on a glass slide to which ID information ID3b2 is attached. The other of the two slices is placed on a glass slide to which ID information ID3b3 is attached.

One slice is cut from the object taken out of the cartridge to which ID information ID3c1 is attached. The slice is placed on a glass slide to which ID information ID3c2 is attached. One slice is cut from the object taken out of the cartridge to which ID information ID3d1 is attached. The slice is placed on a glass slide to which ID information ID3d2 is attached.

Image information Image3d, first ID information, and second ID information are recorded on the recording medium 50. In a first combination of the first ID information and the second ID information, the first ID information is the ID information ID3a1 and the second ID information is the ID information ID3a2 and the ID information ID3a3. In a second combination of the first ID information and the second ID information, the first ID information is the ID information ID3b1 and the second ID information is the ID information ID3b2 and the ID information ID3b3. In a third combination of the first ID information and the second ID information, the first ID information is the ID information ID3c1 and the second ID information is the ID information ID3c2. In a fourth combination of the first ID information and the second ID information, the first ID information is the ID information ID3d1 and the second ID information is the ID information ID3d2.

Two slices are cut from the object taken out of the cartridge to which the ID information ID4a1 is attached. One of the two slices is placed on a glass slide to which ID information ID4a2 is attached. The other of the two slices is placed on a glass slide to which ID information ID4a3 is attached. One slice is cut from the object taken out of the cartridge to which the ID information ID4b1 is attached. The slice is placed on a glass slide to which ID information ID4b2 is attached. One slice is cut from the object taken out of the cartridge to which the ID information ID4c1 is attached. The slice is placed on a glass slide to which ID information ID4c2 is attached.

Image information Image4d, first ID information, and second ID information are recorded on the recording medium 50. In a first combination of the first ID information and the second ID information, the first ID information is the ID information ID4a1 and the second ID information is the ID information ID4a2 and the ID information ID4a3. In a second combination of the first ID information and the second ID information, the first ID information is the ID information ID4b1 and the second ID information is the ID information ID4b2. In a third combination of the first ID information and the second ID information, the first ID information is the ID information ID4c1 and the second ID information is the ID information ID4c2.

In the staining, image information Image3e, first ID information, and second ID information are recorded on the recording medium 50. The first ID information and the second ID information are identical. The ID information ID3a2, the ID information ID3b2, the ID information ID3c2, the ID information ID3d2, the ID information ID3a3, and the ID information ID3b3 are recorded as the first ID information and the second ID information on the recording medium 50.

Also, in the staining, image information Image4e, first ID information, and second ID information are recorded on the recording medium 50. The first ID information and the second ID information are identical. The ID information ID4a2, the ID information ID4b2, the ID information ID4c2, and the ID information ID4a3 are recorded as the first ID information and the second ID information on the recording medium 50.

In the diagnosis, image information Image3f, first ID information, and second ID information are recorded on the recording medium 50. The first ID information and the second ID information are identical. The ID information ID3a2, the ID information ID3b2, the ID information ID3c2, the ID information ID3d2, ID information ID3a3, and the ID information ID3b3 are recorded as the first ID information and the second ID information on the recording medium 50.

Also, in the diagnosis, image information Image4f, first ID information, and second ID information are recorded on the recording medium 50. The first ID information and the second ID information are identical. The ID information ID4a2, ID information ID4b2, the ID information ID4c2, and the ID information ID4a3 are recorded as the first ID information and the second ID information on the recording medium 50.

A user G notices that the pathological specimen to which the ID information ID4b2 is attached is suspicious. Thus, there is a possibility that the mix-up of objects may have occurred. The instruction acceptance unit 61 accepts an instruction for specifying the second ID information ID4b2. The reading unit 70 reads the image information Image4f, the first ID information ID4b2, and the second ID information ID4b2 from the recording medium 50. The display unit 80 displays the image information Image4f, the first ID information ID4b2, and the second ID information ID4b2. The verifier checks whether or not a user E and a user F have performed suspicious behavior on the basis of the image information Image4f. When the cause of the mix-up cannot be found from the image information Image4f, the verifier checks image information of processing directly before the current processing.

As in the first example described above, the verifier sequentially checks the image information Image4e, the image information Image4d, and the image information Image4c. When the cause of the mix-up cannot be found from the image information Image4c, the verifier checks image information of processing directly before the current processing.

The reading unit 70 reads the image information Image3b, the first ID information ID3, and the second ID information ID4b1 from the recording medium 50. The display unit 80 displays the image information Image3b, the first ID information ID3 and the second ID information ID4b1. In the image information Image3b, it is checked that the user B performs an operation different from the cutting operation. It is checked that the user B has different objects before and after an operation such as a telephone call. It is estimated that the object corresponding to the first ID information ID3 has been erroneously put into a cartridge different from the original. It is necessary to identify an object required to be put into the cartridge to which the second ID information ID4b1 is attached.

An object processed at a time point close to a time point at which the object corresponding to the ID information ID4b1 was processed is an object to be checked. Thus, the verifier checks the image information Image4b generated at a time point close to a time point at which the image information Image3b was generated during the cutting. As a result, in the image information Image4b, it is checked that the object corresponding to the ID information ID4 is placed on the glass slide to which the ID information ID3d1 is attached. Originally, this object is required to be placed on the glass slide to which the ID information ID4b1 is attached.

For example, a relationship between ID information of a container arriving at the reception and ID information attached to a cartridge used for an object stored in the container is predefined. Thus, the verifier can determine that a relationship between the ID information ID4 and the ID information ID3d1 is incorrect.

The pathological specimen corresponding to the ID information ID3d1 identified as described above is identified as follows. The instruction acceptance unit 61 accepts an instruction for specifying the first ID information ID3d1. The reading unit 70 reads the image information Image3c, the first ID information ID3d1, and the second ID information ID3d1 from the recording medium 50. The reading unit 70 finally reads the image information Image3f, the first ID information ID3d2, and the second ID information ID3d2 from the recording medium 50 by processing similar to that of the first example described above. There is a possibility that the pathological specimen to which the ID information ID3d2 is attached may have been mistaken for the pathological specimen to which the ID information ID4b2 is attached.

A comparison is made between the pathological specimen to which the ID information ID3c2 is attached and the pathological specimen to which the ID information ID3d2 is attached. These pathological specimens should be cut from the same portion and have common features such as structures. Also, a comparison is made between the pathological specimen to which the ID information ID4a2 is attached and the pathological specimen to which the ID information ID4b2 is attached. These pathological specimens should be cut from the same portion and have common features such as structures. As a result of the above, it is found that the mix-up of the objects has occurred between the pathological specimen to which the ID information ID3d2 is attached and the pathological specimen to which the ID information ID4b2 is attached.

As described above, image information to be checked is identified on the basis of ID information corresponding to objects that are likely to be mixed up. The user can check the cause of the mix-up by checking image information that is processing information.

Verification of a cause of an operation error other than the mix-up of objects may be performed on the basis of image information. For example, when a cutting direction in the cutting is different from a predefined direction, a pathological specimen suitable for observation is not made. Thus, the checking of the cutting direction may be performed on the basis of the image information generated during the cutting. When a reagent has degraded in staining, no suitable staining is performed. Thus, the checking of the reagent may be performed on the basis of image information generated during the staining. When staining time in the staining is different from a predefined time, no suitable staining is performed. Thus, the checking of the staining time may be performed on the basis of image information generated during the staining.

### Sixth Embodiment

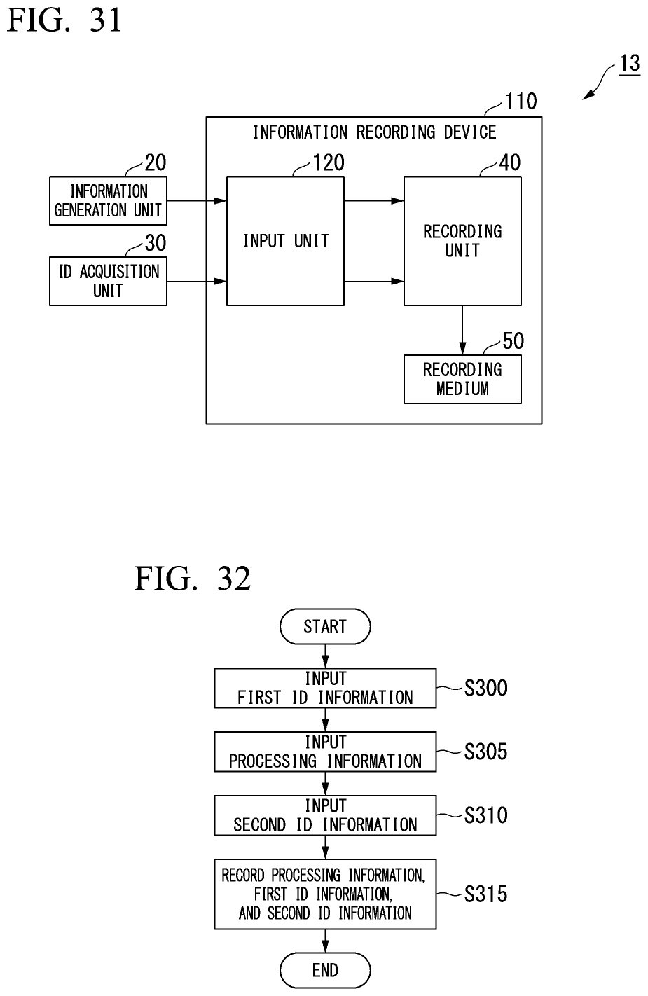

FIG. 31 shows a configuration of an information recording system 13 according to a sixth embodiment of the present invention. Differences from the configuration shown in FIG. 1 will be described in terms of the configuration shown in FIG. 31.

As shown in FIG. 31, the information recording system 13 includes an information generation unit 20, an ID acquisition unit 30, and an information recording device 110. The information generation unit 20 is similar to the information generation unit 20 shown in FIG. 1. The ID acquisition unit 30 is similar to the ID acquisition unit 30 shown in FIG. 1. In the information recording system 13 shown in FIG. 31, the recording unit 40 and the recording medium 50 in the information recording system 10 shown in FIG. 1 are changed to the information recording device 110.

The information recording device 110 includes a recording unit 40, a recording medium 50, and an input unit 120. The recording unit 40 is similar to the recording unit 40 shown in FIG. 1. The recording medium 50 is similar to the recording medium 50 shown in FIG. 1. Processing information generated by the information generation unit 20 and ID information acquired by the ID acquisition unit 30 are input to the input unit 120. For example, at least one of the information generation unit 20 and the ID acquisition unit 30 and the information recording device 110 are connected by a cable. In this case, the input unit 120 is an input terminal to which the cable is connected. At least one of the information generation unit 20 and the ID acquisition unit 30 and the information recording device 110 may be connected wirelessly. In this case, the input unit 120 is a wireless communication unit configured to perform wireless communication with at least one of the information generation unit 20 and the ID acquisition unit 30.

The recording medium 50 may be disposed outside the information recording device 110. The recording medium 50 may be attachable to the information recording device 110 and removable from the information recording device 110. The information recording device 110 may have a network interface and the information recording device 110 may be connected to the recording medium 50 via a network. The information recording device 110 may have a wireless communication interface and the information recording device 110 may be connected to the recording medium 50 by wireless communication.

The information recording device 110 may read a program and execute the read program. That is, the functions of the information recording device 110 may be implemented by software. This program includes commands for defining the operation of the recording unit 40. For example, this program may be provided by a “computer-readable recording medium” such as a flash memory. Also, the above-described program may be transmitted from a computer having a storage device or the like in which the program is stored to the information recording device 110 via a transmission medium or transmission waves in the transmission medium. The “transmission medium” for transmitting the program refers to a medium having an information transmission function, for example, a network (a communication network) such as the Internet or a communication circuit (a communication line) such as a telephone circuit. Also, the above-described program may be a program for implementing some of the above-described functions. Further, the above-described program may be a program capable of implementing the above-described function in combination with a program already recorded on the computer, i.e., a so-called differential file (differential program).

Except for the points described above, the configuration shown in FIG. 31 is similar to the configuration shown in FIG. 1.

FIG. 32 shows order of a process to be performed by the information recording device 110. The order of the process to be performed by the information recording device 110 will be described with reference to FIG. 32.

First ID information acquired by the ID acquisition unit 30 is input to the input unit 120 (step S300 (input step)). After step S300, processing information generated by the information generation unit 20 is input to the input unit 120 (step S305 (input step)). After step S305, second ID information acquired by the ID acquisition unit 30 is input to the input unit 120 (step S310 (input step)). After step S310, the recording unit 40 associates the first ID information and the second ID information with the processing information, and records the processing information, the first ID information, and the second ID information on the recording medium (step S315 (recording step)).

As described above, the processing information, the first ID information, and the second ID information are input to the input unit 120. The recording unit 40 associates the first ID information and the second ID information with the processing information, and records the processing information, the first ID information, and the second ID information on the recording medium 50. Thus, the information recording device 110 can support verification of the validity of a processing situation.

The matters disclosed in the first to fifth modified examples of the first embodiment may be similarly applied to the information recording system 13 of the sixth embodiment.

For example, the information recording system 13 may include the instruction acceptance unit 60 shown in FIG. 6. That is, a start instruction and an end instruction accepted by the instruction acceptance unit 60 may be input to the input unit 120. The information recording device 110 may include the instruction acceptance unit 60 shown in FIG. 6.

In the information recording system 13, the ID acquisition unit 30 may be changed to the ID acquisition unit 30b shown in FIG. 8. That is, the first ID information acquired by the first ID acquisition unit 300 and the second ID information acquired by the second ID acquisition unit 301 may be input to the input unit 120.

In the information recording system 13, the ID acquisition unit 30 may be changed to the ID acquisition unit 30c shown in FIG. 9. That is, the ID information acquired by the image processing unit 303 may be input to the input unit 120. The ID acquisition unit 30c may not include the image processing unit 303. Therefore, image information generated by the camera 302 may be input as ID information to the input unit 120.

In the information recording system 13, the information generation unit 20 may be changed to the information generation unit 20d shown in FIG. 10 and the ID acquisition unit 30 may be changed to the ID acquisition unit 30c shown in FIG. 10. That is, processing information may be input from the camera 302 or the image processing unit 303 to the input unit 120. The ID information acquired by the image processing unit 303 may be input to the input unit 120. The ID acquisition unit 30c may not include the image processing unit 303. Therefore, image information generated by the camera 302 may be input as ID information to the input unit 120.

In the information recording system 13, the ID acquisition unit 30 may be changed to the ID acquisition unit 30e shown in FIG. 15. That is, the ID information recognized by the sound processing unit 305 may be input to the input unit 120.

### Seventh Embodiment

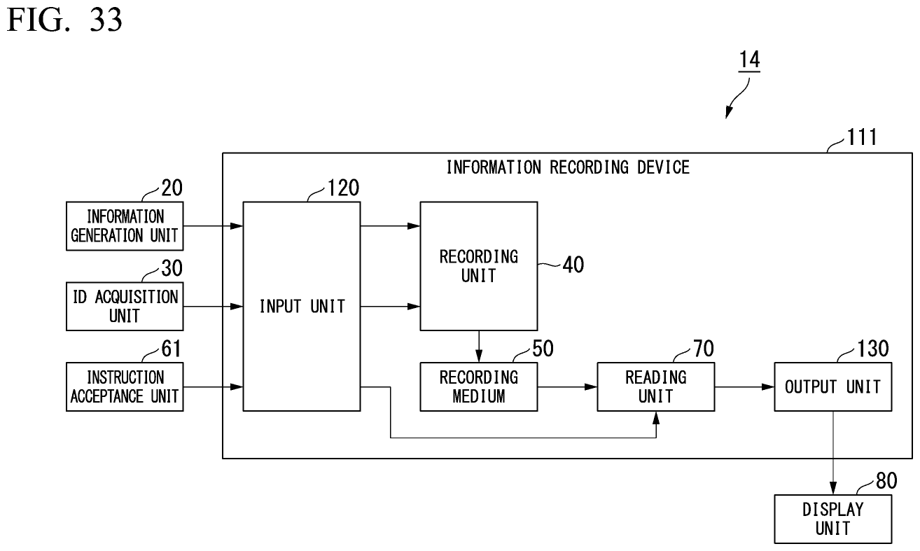

FIG. 33 shows a configuration of an information recording system 14 according to a seventh embodiment of the present invention. Differences from the configuration shown in FIG. 31 will be described in terms of the configuration shown in FIG. 33.

In the information recording system 14, the information recording device 110 shown in FIG. 31 is changed to an information recording device 111. The information recording system 14 further includes an instruction acceptance unit 61 and a display unit 80.

The instruction acceptance unit 61 is similar to the instruction acceptance unit 61 shown in FIG. 16. An instruction accepted by the instruction acceptance unit 61 is input to the input unit 120. The information recording device 111 includes a reading unit 70 and an output unit 130 in addition to the configuration of the information recording device 110 shown in FIG. 31. The reading unit 70 reads processing information associated with at least one piece of first ID information and second ID information corresponding to the instruction input to the input unit 120 and the first ID information and the second ID information associated with the processing information from the recording medium 50.

The output unit 130 outputs the processing information, the first ID information, and the second ID information read by the reading unit 70. For example, the display unit 80 and the information recording device 111 are connected by a cable. In this case, the output unit 130 is an output terminal to which the cable is connected. The display unit 80 and the information recording device 111 may be connected wirelessly. In this case, the output unit 130 is a wireless communication unit configured to perform wireless communication with the display unit 80. The display unit 80 displays the processing information, the first ID information, and the second ID information output by the output unit 130. The information recording device 111 may include the instruction acceptance unit 61.

Except for the points described above, the configuration shown in FIG. 33 is similar to the configuration shown in FIG. 31.

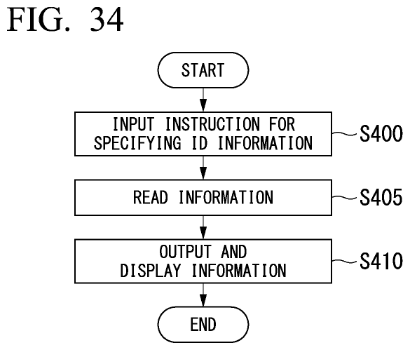

The information recording device 111 performs a process shown in FIG. 32. After the process shown in FIG. 32 is completed, the information recording device 111 performs a process shown in FIG. 34. FIG. 34 shows order of the process to be performed by the information recording device 111. The order of the process to be performed by the information recording device 111 will be described with reference to FIG. 34.

The instruction acceptance unit 61 accepts an instruction for specifying at least one piece of first ID information and second ID information. At this time, the instruction is input to the input unit 120 (step S400 (instruction input step)). After step S400, the reading unit 70 reads processing information associated with at least one piece of the first ID information and the second ID information corresponding to the instruction input to the input unit 120 from the recording medium 50. Also, the reading unit 70 reads the first ID information and the second ID information associated with the processing information from the recording medium 50 (step S405 (reading step)). After step S405, the output unit 130 outputs the processing information, the first ID information, and the second ID information read by the reading unit 70. The display unit 80 displays the processing information, the first ID information, and the second ID information output by the output unit 130 (step S410 (output step and display step)).

As in the third embodiment, the instruction acceptance unit 61 may accept an instruction for specifying processing information. The instruction accepted by the instruction acceptance unit 61 may be input to the input unit 120. The reading unit 70 may read the processing information corresponding to the instruction input to the input unit 120 and first ID information and second ID information associated with the processing information from the recording medium 50.

As described above, the information recording system 14 includes the instruction acceptance unit 61 and the information recording device 111 includes the reading unit 70 and the output unit 130. Thereby, the information recording device 111 can acquire information associated with the ID information or the processing information corresponding to the instruction accepted by the instruction acceptance unit 61 from the recording medium 50.

### Eighth Embodiment

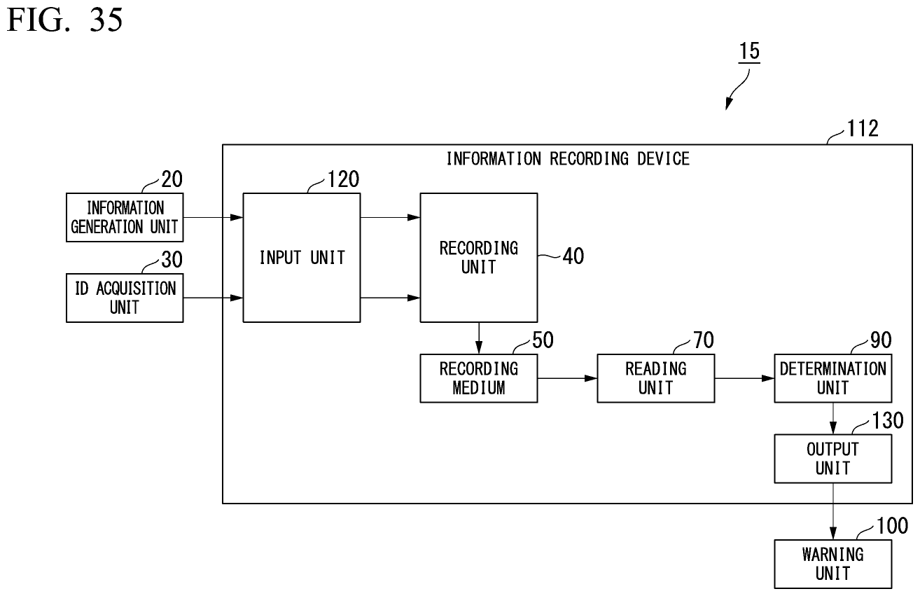

FIG. 35 shows a configuration of an information recording system 15 according to an eighth embodiment of the present invention. Differences from the configuration shown in FIG. 31 will be described in terms of the configuration shown in FIG. 35.

In the information recording system 15, the information recording device 110 shown in FIG. 31 is changed to an information recording device 112. The information recording system 15 further includes a warning unit 100.

The information recording device 112 has a reading unit 70, a determination unit 90, and an output unit 130 in addition to the configuration of the information recording device 110 shown in FIG. 31. A reading unit 70 is similar to the reading unit 70 shown in FIG. 25. The determination unit 90 determines whether or not at least one piece of processing information, first ID information, and second ID information recorded on a recording medium 50 matches a condition predefined as a condition to be satisfied by the processing information, the first ID information, and the second ID information. The determination unit 90 generates control information according to a determination result. The output unit 130 outputs the control information generated by the determination unit 90. The warning unit 100 issues a warning on the basis of the control information when at least one piece of the processing information, the first ID information, and the second ID information recorded on the recording medium 50 does not match the above-described condition.

Except for the points described above, the configuration shown in FIG. 35 is similar to the configuration shown in FIG. 31.

The information recording device 112 performs the process shown in FIG. 32. After the process shown in FIG. 32 is completed, the information recording device 112 performs a process similar to the process shown in FIG. 26. In step S240 shown in FIG. 26, when at least one piece of the information read from the recording medium 50 does not match the condition indicated by the condition information, the determination unit 90 generates control information to cause the warning unit 100 to output a warning. The output unit 130 outputs the control information generated by the determination unit 90. The warning unit 100 outputs the warning on the basis of the control information.

As described above, the information recording system 15 includes the warning unit 100 and the information recording device 112 includes the determination unit 90 and the output unit 130. Thereby, the information recording device 112 can reduce the number of operation errors that will occur. When an operation error has occurred, the information recording device 112 can support verification of the validity of a processing situation as in the case of the information recording device 110 of the sixth embodiment.

While preferred embodiments of the invention have been described and shown above, it should be understood that these are exemplars of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An information recording system comprising:
an information generation unit including at least one of a sensor and a camera and configured to generate processing information for identifying processing to be performed on an object for inspection;
an ID acquisition unit including at least one of a communicator and a scanner and configured to acquire ID information attached to at least one of the object and a physical object for storing or supporting the object; and
a recording unit including a processor and configured to associate first ID information and second ID information with the processing information and record the processing information, the first ID information, and the second ID information on a recording medium, the first ID information being the ID information acquired by the ID acquisition unit before the processing is performed, the second ID information being the ID information acquired by the ID acquisition unit after the processing is performed.

2. The information recording system according to claim 1, wherein the information generation unit is mounted on an instrument for use in the processing and outputs the processing information when the use of the instrument has been detected.

3. The information recording system according to claim 2, wherein the instrument is used to make pathological specimens.

4. The information recording system according to claim 1,
wherein the information generation unit includes a camera configured to generate image information by photographing a state of the processing, and
the recording unit records the image information generated by the camera as the processing information on the recording medium.

5. The information recording system according to claim 1, further comprising a first instruction acceptance unit including at least one of an operation device and a communicator and configured to accept a start instruction related to a start of the processing and an end instruction related to an end of the processing,
wherein the first ID information is the ID information acquired by the ID acquisition unit at a timing associated with a timing at which the start instruction is accepted, and
the second ID information is the ID information acquired by the ID acquisition unit at a timing associated with a timing at which the end instruction is accepted.

6. The information recording system according to claim 1, wherein the ID acquisition unit includes
a first ID acquisition unit including at least one of a communicator and a scanner and configured to acquire the first ID information; and
a second ID acquisition unit, different from the first ID acquisition unit, including at least one of a communicator and a scanner and configured to acquire the second ID information.

7. The information recording system according to claim 1,
wherein the ID acquisition unit includes a camera configured to generate image information by photographing at least one of the object and the physical object, and
the recording unit records the image information generated by the camera as the ID information on the recording medium.

8. The information recording system according to claim 1,
wherein the ID acquisition unit includes
a camera configured to generate image information by photographing at least one of the object and the physical object, and
an image processing unit including a processor and configured to acquire the ID information by performing image processing on the image information generated by the camera.

9. The information recording system according to claim 8,
wherein the image processing unit acquires the first ID information from a first region in the image information, and
the image processing unit acquires the second ID information from a second region different from the first region in the image information.

10. The information recording system according to claim 8,
wherein, when a first motion of the ID information in the image information has been recognized, the image processing unit acquires the ID information as the first ID information,
when a second motion of the ID information in the image information has been recognized, the image processing unit acquires the ID information as the second ID information, and
the second motion is different from the first motion.

11. The information recording system according to claim 1,
wherein a radio tag on which the ID information is recorded is attached to at least one of the object and the physical object, and
the ID acquisition unit is a wireless communicator configured to receive the ID information from the radio tag.

12. The information recording system according to claim 1, wherein the ID acquisition unit includes
a sound acquisition unit including a microphone and configured to acquire sound information based on sound uttered by a user who performs the process; and
a sound processing unit including a processor and configured to recognize the ID information on the basis of the sound information acquired by the sound acquisition unit.

13. The information recording system according to claim 1,
wherein the information generation unit generates the processing information corresponding to each of a plurality of types of the processing,
the ID acquisition unit acquires the first ID information and the second ID information corresponding to each of the plurality of types of the processing,
the recording unit associates the first ID information and the second ID information corresponding to each of the plurality of types of the processing with the processing information corresponding to each of the plurality of types of the processing, and
the recording unit records the processing information, the first ID information, and the second ID information corresponding to each of the plurality of types of the processing on the recording medium.

14. The information recording system according to claim 1, wherein the recording unit further associates time point information indicating a time point at which the processing was performed with the processing information and records the time point information on the recording medium.

15. The information recording system according to claim 1, further comprising:
a second instruction acceptance unit including at least one of an operation device and a communicator and configured to accept an instruction for specifying at least one piece of the first ID information and the second ID information; and
a reading unit including a processor and configured to read the processing information associated with at least one piece of the first ID information and the second ID information corresponding to the instruction from the recording medium and read the first ID information and the second ID information associated with the processing information from the recording medium.

16. The information recording system according to claim 15,
wherein, when the second instruction acceptance unit has accepted an instruction for specifying the second ID information, the reading unit reads first processing information associated with the second ID information corresponding to the instruction from the recording medium and reads the first ID information and the second ID information associated with the first processing information from the recording medium, and
the reading unit further reads second processing information associated with the second ID information identical to the first ID information associated with the first processing information from the recording medium and reads the first ID information and the second ID information associated with the second processing information from the recording medium.

17. The information recording system according to claim 15,
wherein, when the second instruction acceptance unit has accepted an instruction for specifying the first ID information, the reading unit reads first processing information associated with the first ID information corresponding to the instruction from the recording medium and reads the first ID information and the second ID information associated with the first processing information from the recording medium, and
the reading unit further reads second processing information associated with the first ID information identical to the second ID information associated with the first processing information from the recording medium and reads the first ID information and the second ID information associated with the second processing information from the recording medium.

18. The information recording system according to claim 1, further comprising:
a third instruction acceptance unit including at least one of an operation device and a communicator and configured to accept an instruction for specifying the processing information; and
a reading unit including a processor and configured to read the processing information corresponding to the instruction from the recording medium and read the first ID information and the second ID information associated with the processing information from the recording medium.

19. The information recording system according to claim 18,
wherein, when the third instruction acceptance unit has accepted an instruction for specifying first processing information, the reading unit reads the first processing information corresponding to the instruction from the recording medium and reads the first ID information and the second ID information associated with the first processing information from the recording medium, and
the reading unit further reads second processing information associated with the second ID information identical to the first ID information associated with the first processing information from the recording medium and reads the first ID information and the second ID information associated with the second processing information from the recording medium.

20. The information recording system according to claim 18,
wherein, when the third instruction acceptance unit has accepted an instruction for specifying first processing information, the reading unit reads the first processing information corresponding to the instruction from the recording medium and reads the first ID information and the second ID information associated with the first processing information from the recording medium, and
the reading unit further reads second processing information associated with the first ID information identical to the second ID information associated with the first processing information from the recording medium and reads the first ID information and the second ID information associated with the second processing information from the recording medium.

21. The information recording system according to claim 15, further comprising a display configured to display the processing information, the first ID information, and the second ID information read by the reading unit.

22. The information recording system according to claim 1, further comprising:
a determination unit including a processor and configured to determine whether or not at least one piece of the processing information, the first ID information, and the second ID information recorded on the recording medium matches a condition predefined as a condition to be satisfied by the processing information, the first ID information, and the second ID information; and
a warning unit including at least one of a display and a speaker and configured to issue a warning when at least one piece of the processing information, the first ID information, and the second ID information recorded on the recording medium does not match the condition.

23. An information recording device comprising:
an input unit including at least one of an input terminal and a communicator to which processing information generated by an information generation unit including at least one of a sensor and a camera and ID information acquired by an ID acquisition unit including at least one of a communicator and a scanner are input, the information generation unit being configured to generate the processing information for identifying processing to be performed on an object for inspection, the ID acquisition unit being configured to acquire the ID information attached to at least one of the object and a physical object for storing or supporting the object; and
a recording unit including a processor and configured to associate first ID information and second ID information with the processing information and record the processing information, the first ID information, and the second ID information on a recording medium, the first ID information being the ID information acquired by the ID acquisition unit before the processing is performed, the second ID information being the ID information acquired by the ID acquisition unit after the processing is performed.

24. An information recording method comprising:
an information generation step in which an information generation unit including at least one of a sensor and a camera generates processing information for identifying processing to be performed on an object for inspection;
an ID acquisition step in which an ID acquisition unit including at least one of a communicator and a scanner acquires ID information attached to at least one of the object and a physical object for storing or supporting the object; and
a recording step in which a recording unit including a processor associates first ID information and second ID information with the processing information and records the processing information, the first ID information, and the second ID information on a recording medium, the first ID information being the ID information acquired by the ID acquisition unit before the processing is performed, the second ID information being the ID information acquired by the ID acquisition unit after the processing is performed.

25. An information recording method comprising:
an input step in which processing information generated by an information generation unit including at least one of a sensor and a camera and ID information acquired by an ID acquisition unit including at least one of a communicator and a scanner are input to an input unit including at least one of an input terminal and a communicator, the information generation unit being configured to generate the processing information for identifying processing to be performed on an object for inspection, the ID acquisition unit being configured to acquire the ID information attached to at least one of the object and a physical object for storing or supporting the object; and
a recording step in which a recording unit including a processor associates first ID information and second ID information with the processing information and records the processing information, the first ID information, and the second ID information on a recording medium, the first ID information being the ID information acquired by the ID acquisition unit before the processing is performed, the second ID information being the ID information acquired by the ID acquisition unit after the processing is performed.
